(19) United States
(12) Patent Application Publication
(10) Pub. No.: US 2024/0261779 A1
(43) Pub. Date: Aug. 8, 2024

(54) SYSTEMS AND METHODS FOR FLUID SAMPLE COLLECTION AND TESTING

(71) Applicant: Marshall Venture Partners LLC, Marshall, TX (US)

(72) Inventors: Alan R Loudermilk, Marshall, TX (US); Herbert Hunt Allred, Dallas, TX (US); Kevin Fuller, Chaska, MN (US); Wayde J Altendorf, Hickson, ND (US)

(73) Assignee: Marshall Venture Partners LLC, Marshall, TX (US)

(21) Appl. No.: 18/105,241

(22) Filed: Feb. 2, 2023

(51) Int. Cl. B01L 3/00; G01N 1/10

(52) U.S. Cl. CPC B01L 3/5023; G01N 1/10; B01L 2200/0684; B01L 2200/0689; B01L 2300/069; B01L 2300/0825; B01L 2300/0832; B01L 2400/0481; G01N 2021/7759; G01N 21/78

(57) ABSTRACT

Improved fluid collection and test systems and methods provide for a fluid specimen to be collected via a swab with an absorbent sponge-like tip and have a first portion of the collected specimen dispensed into an external vessel (such as a cassette or holder for testing) as a first step, and a second portion of the collected specimen dispenses into a specimen container as a second step. The swab may be compressed by downward compression of the swab in a compression tube with a movable venting gasket/seal having one or more cross members impeding the swab but allowing passage of fluid specimen into an external test vessel or specimen container. Improved swab assemblies have analyte test holders for holding one or more analyte test strips that may be inserted into a “top” end of the swab assembly that is distal from a “bottom” end of the swab assembly that has a sponge-like material for collecting a fluid specimen.

### FIELD OF THE INVENTION

The present invention relates to substance collection and testing particularly of fluid samples, and more particularly the present invention relates to a device that tests a fluid sample for the presence or absence of at least one analyte with improved fluid extraction and air flow characteristics, preferably (but optionally) securing a separate fluid sample for later confirmation, and preferably (but optionally) providing positive identification of an individual associated with the sample. The “dual swab” embodiments referenced herein are sometimes referred to herein as a “Dual Swab Device.” Embodiments thereof having only a “single swab” are referred to herein as a “Single Swab Device.” Both Dual Swab Devices and Single Swab Devices are within the scope of the present invention and preferably include one or more lateral flow assay strips integrally in a swab stem assembly and optionally also in a test strip chamber, and preferably may have dual or single test strip cassettes in the test strip chamber as herein after described. A single swab “Vial Side Only Device” also is within the scope of the present invention.

### BACKGROUND OF THE INVENTION

Reference is made to co-pending US App. Ser. Nos. 10/792,175, 15/417,905, 15/418,044, 17/797,171, and 17/896,790, which are hereby incorporated by reference (“Referenced Patent Documents”).

Drug and other analyte testing has become ubiquitous in modem society. In homes, doctors' offices, law enforcement vehicles and offices, athletic facilities, and the workplace, effective, inexpensive and reliable testing devices have been sought. There is also a growing need for devices to test bodily fluids for substances that may assist in the diagnosis or management of diseases and other medical conditions.

The marketplace responded and is now replete with many devices directed to the testing of blood, urine or saliva. However, these devices may require a series of tests involving the shifting of the fluid sample being tested to different containers and/or the removal of the fluid sample to distant locations. These devices may also require the test administrator to handle the test subject's bodily fluids, incurring a danger of disease exposure.

Once an initial test result has been obtained, further testing of the same fluid sample to confirm or refine the initial test result is often required. For a membrane test strip device, the fluid sample may not even be retained once the initial result is obtained, necessitating retention of a split sample. The need to retain a split sample incurs the risk that a sample could be lost, mislabeled, or contaminated.

Oftentimes, the chain of custody associated with a test sample imbues the results with doubt, as the fluid sample may become contaminated, misplaced or a different fluid sample may be substituted entirety. In many instances, identification of the test subject associated with the fluid sample is critically dispositive.

Prior art testing devices include those disclosed in U.S. Pat. Nos. 7,879,623 and 8,940,527, both entitled “Integrated Device for Analyte, Testing, Confirmation, and Donor Identity Verification” and both identifying Raouf A. Guirguis as the sole inventor. U.S. Pat. Nos. 7,879,623 and 8,940,527 are both hereby incorporated by reference. The patents disclose an apparatus for fluid sample collection and analyte testing, including a single sample receiving member and at least one membrane test strip, and optionally a sample retention member, fingerprint acquisition pad, and/or fluid collector. It also provides a fluid collection apparatus having an absorbent material, compression element, and closure element, and optionally a lid that allows the apparatus to be used in conjunction with a fluid container. Also provided are methods of collecting, testing, and retaining a fluid sample and verifying the identity of one or more individuals associated with the sample, such as the test subject, test administrator, and/or witnesses. The components for collecting, testing, and retaining a fluid sample are in fluid communication with the other components of the testing device.

There is also a growing need for devices directed to testing for contaminants that may be found in food or water, such as pollutants, allergens, and harmful microbes. In some instances, it may be desirable to retain a fluid sample for confirmation testing or further analysis, retain a split fluid sample of the original sample for confirmation testing or further analysis, or to provide positive identification of the test administrator.

The Department of Transportation's (DOT) rule, 49 C.F.R. Part 40, describes required procedures for conducting workplace drug and alcohol testing for the Federally regulated transportation industry. Within this rule, definitions for split sample and split sample collection are provided. Split specimen is defined as, in drug testing, a part of the urine specimen that is sent to a first laboratory and retained unopened, and which is transported to a second laboratory in the event that the employee requests that it be tested following a verified positive test of the primary specimen or a verified adulterated or substituted test result. Split specimen collection is defined as a collection in which the urine collected is divided into two separate specimen bottles, the primary specimen (Bottle A) and the split specimen (Bottle B).

A need exists in the industry for improved fluid collection systems and methods by which, for example, a fluid specimen may be collected via a swab and have a first portion of the collected specimen dispensed into an external vessel (such as a cassette or holder for testing) as a first step, and a second portion of the collected specimen dispenses into a specimen container as a second step.

A need also exists in the industry for improved methods of compressing fluid specimens from a swab in which the swab is compressed by downward compression of the swab in a compression tube with a movable venting gasket/seal having one or more cross members impeding the swab but allowing passage of fluid specimen into an external test vessel, specimen container, or other location external to the compression tube.

A need also exists in the industry for improved swab assemblies having analyte test holders for holding one or more analyte test strips that may be inserted into a “top” end of the swab assembly that is distal from a “bottom” end of the swab assembly that has a sponge-like material for collecting a fluid specimen.

Also incorporated by reference herein are the disclosures of U.S. Pat. Nos. 9,414,813 and 10,035,146.

### SUMMARY OF THE INVENTION

Embodiments claimed herein are expressly directed to devices and methods preferably including having a fluid specimen collected via a swab and have a first portion of the collected specimen dispensed into an external test vessel as a first step, and a second portion of the collected specimen dispenses into a specimen container as a second step, wherein the specimen preferably is dispensed via the swab being compressed by downward compression of the swab in a compression tube with a movable venting gasket having a tapered dispensing end, and preferably also having one or more cross members impeding the swab (and assisting in swab compression) but having preferably a plurality of openings allowing passage of fluid specimen into an external test vessel, specimen container, or other location external to the compression tube. Embodiments claimed herein are also expressly directed to improved swab assemblies having analyte test holders for holding one or more analyte test strips that may be inserted into an end of the swab assembly that is distal from an end of the swab assembly that has a sponge-like material for collecting a fluid specimen. Embodiments described herein are expressly intended for combined use as will be described elsewhere herein.

In accordance with certain preferred embodiments of the present invention, a fluid sample testing apparatus has: a housing with a test chamber and a first fluid collector tube of a first diameter and a second fluid collector tube of a second diameter, wherein the test chamber is in fluid communication with the first fluid collector tube; a sample holding container in fluid communication with the second fluid collector tube; and first and second fluid collectors, wherein the first fluid collector is adapted for insertion into the first fluid collector tube and upon insertion into the first fluid collector tube pressure is generated to release fluid from the first fluid collector into the test chamber via an opening between the test chamber and the first fluid collector tube, wherein air passes outside of the apparatus from the test chamber via an opening from the test chamber into the first fluid collector tube, and wherein the second fluid collector is adapted for insertion into the second fluid collector tube concurrently with insertion of the first fluid collector into the first fluid collector tube and upon insertion into the second fluid collector tube pressure is generated to release fluid from the second fluid collector into the sample holding container, wherein air passes outside of the apparatus from the second fluid collector tube. Preferably, such embodiments include a movable venting gasket/seal as described elsewhere herein, and/or a swab assembly having an analyte test strip holder inserted into a “top” end of the swab assembly as described elsewhere herein.

The apparatus may have the first fluid collector having an upper sealing portion such that upon substantially complete insertion into the first fluid collector tube the upper sealing portion seals the opening between the test chamber and the first fluid collector swab tube. The apparatus may have the second fluid collector tube having a shoulder portion in proximity to the sample holding container of a diameter smaller than the second diameter, wherein the second fluid collector has a lower sealing portion such that upon substantially complete insertion into the second fluid collector tube the lower sealing portion seals the sample holding container from an upper portion of the second fluid collector tube. The first and second fluid collectors may comprise individual single swabs coupled together with a locked cap. The second diameter preferably is greater than the first diameter. The test chamber preferably has a top cap connected to the housing that is sealed from outside of the apparatus, preferably in an air and water tight manner such as by sonic welding. Preferably, such embodiments include a movable venting gasket/seal as described elsewhere herein, and/or a swab assembly having an analyte test strip holder inserted into a “top” end of the swab assembly as described elsewhere herein.

Embodiments of the invention preferably further include a cassette for holding one or a plurality of test strips, wherein the cassette includes one or more channels for holding the one or more test strips and includes at least a first channel for holding a first strip, the first channel having a plurality of projections from each of two opposite side walls of the first channel, wherein the plurality of projections define a center portion in which the first strip is positioned, wherein the plurality of projections position the first strip to reduce or prevent contact between the first strip and the side walls of the first channel, wherein the first channel also has a floor having a plurality of raised portions, wherein the plurality of raised portions position the first strip to reduce or prevent contact between the first strip and the floor of the first channel. Bumps on the front face of the cassette also are provided in certain embodiments, as are side legs that are tapered or have a smaller dimension at a bottom portion compared to a larger dimension at an upper portion. Preferably, such embodiments include a movable venting gasket/seal as described elsewhere herein, and/or a swab assembly having an analyte test strip holder inserted into a “top” end of the swab assembly as described elsewhere herein.

Embodiments of the present invention also include a fluid sample testing apparatus having a housing with a test chamber and a first fluid collector tube of a first diameter and a second fluid collector tube of a second diameter, wherein the test chamber is in fluid communication with the first fluid collector tube; a sample holding container in fluid communication with the second fluid collector tube; first and second fluid collectors, wherein the first fluid collector is adapted for insertion into the first fluid collector tube and upon insertion into the first fluid collector tube pressure is generated to release fluid from the first fluid collector into the test chamber via an opening between the test chamber and the first fluid collector tube, wherein air passes outside of the apparatus from the test chamber via an opening from the test chamber into the first fluid collector tube, and wherein the second fluid collector is adapted for insertion into the second fluid collector tube concurrently with insertion of the first fluid collector into the first fluid collector tube and upon insertion into the second fluid collector tube pressure is generated to release fluid from the second fluid collector into the sample holding container; and a gasket positioned in the second fluid collector tube having an upper portion for engaging a wall of the second fluid collector tube and a lower portion of smaller diameter than the upper portion for insertion into an opening of the sample holding container, wherein the gasket is positioned at an upper location in the second fluid collector tube upon insertion of the second fluid collector into the second fluid collector tube, wherein the gasket is positioned at a lower location in the second fluid collector tube after insertion of the second fluid collector into the second fluid collector tube and at the lower location the gasket forms a seal with the sample holding container, wherein air passes outside of the apparatus from the second fluid collector tube before the gasket is positioned at the lower location. Preferably, such embodiments include a movable venting gasket/seal as described elsewhere herein, and/or a swab assembly having an analyte test strip holder inserted into a “top” end of the swab assembly as described elsewhere herein.

The first fluid collector may have an upper sealing portion such that upon substantially complete insertion into the first fluid collector tube the upper sealing portion seals an opening between the test chamber and the first fluid collector swab tube. The apparatus may have the first fluid collector having a lower sealing portion adapted to form a sliding seal between the lower sealing portion and a surface wall of the first fluid collector tube as the first fluid collector is inserted into the first fluid collector tube. The upper sealing portion may be adapted to form a fixed seal for the opening between the test chamber and the first fluid collector tube and have a shape different from the lower sealing portion. The upper sealing portion may form a stopper seal for the opening between the test chamber and the first fluid collector tube, and the lower sealing portion may form a syringe plunger seal between the lower sealing portion and a surface wall of the first fluid collector tube. The lower sealing portion may be formed to have a plurality of spaced apart o-ring portions to form the syringe plunger seal. The lower sealing portion may be formed to have a textured surface to form the syringe plunger seal. Preferably, such embodiments include a movable venting gasket/seal as described elsewhere herein, and/or a swab assembly having an analyte test strip holder inserted into a “top” end of the swab assembly as described elsewhere herein.

In particular, preferred embodiments of the present invention include a Vial-Side Only Device (as described hereinafter) particularly such having a swab assembly with one or more lateral flow test strips in the stem of the swab assembly so that the presence of absence of one or more analytes may be detected with one or more lateral flow strips, each of which may detect one or more analytes, with the swab assembly providing an absorbent member which upon compression results in a portion of the fluid specimen in a vial, which may be used for confirmation or other testing based on the fluid specimen deposited in the vial. Preferably, such embodiments include a movable venting gasket/seal as described elsewhere herein, and/or a swab assembly having an analyte test strip holder inserted into a “top” end of the swab assembly as described elsewhere herein.

Accordingly, it is an object of the present invention to provide single and dual swab fluid collection and testing devices having improved air venting/air flow characteristics to improve fluid transfer into a test chamber via a channel and optionally into a confirmation vial.

It is another object of the present invention to provide single and dual swab fluid collection and testing devices having improved user experience by having reduced back pressure when swab assemblies are inserted into and pressured downward in a preferably locked position.

It is yet another object of the present invention to provide swab assemblies having a stopper type upper seal and a syringe type lower seal.

It further is an object of the present invention to provide a fluid collection apparatus having a funnel shaped gasket in a first position whereby a swab may be initially compressed, and upon pressure build-up move to a second position whereby the gasket seals to the housing and vial, wherein prior to being in the second position air may vent out of the apparatus.

It further is an object of the present invention to provide a strip holding cassette for use in testing fluid samples having one or more channels with side projections and floor bumps that position a strip so as to reduce or prevent contact of the strip with side walls of the channel or a floor of the channel.

It further is an object of the present invention to provide improved fluid collection systems and methods by which, for example, a fluid specimen may be collected via a swab and have a first portion of the collected specimen dispensed into an external vessel (such as a cassette or holder for testing) as a first step, and a second portion of the collected specimen dispenses into a specimen container as a second step.

It is further an object of the present invention to provide improved methods of compressing fluid specimens from a swab in which the swab is compressed by downward compression of the swab in a compression tube with a movable venting gasket/seal having one or more cross members impeding the swab but allowing passage of fluid specimen into an external test vessel, specimen container, or other location external to the compression tube.

Finally, it is an object of the present invention to provide improved swab assemblies having analyte test holders for holding one or more analyte test strips that may be inserted into a “top” end of the swab assembly that is distal from a “bottom” end of the swab assembly that has a sponge-like material for collecting a fluid specimen.

### BRIEF DESCRIPTION OF THE DRAWINGS

The above objects and other advantages of the present invention will become more apparent by describing in detail the preferred embodiments of the present invention with reference to the attached drawings in which:

FIGS. 1-3 illustrate overall device views of a fluid sample collection and testing device in accordance with certain preferred embodiments of the present invention;

FIG. 4 illustrates fluid communication between a swab tube and a test chamber and between a swab tube and a confirmation vial in accordance with certain preferred embodiments of the present invention;

FIGS. 5A and 5B illustrate a top cap preferably sonic weldable in accordance with certain preferred embodiments of the present invention;

FIGS. 6A-6C illustrate a swab stem and a swab assembly in accordance with certain preferred embodiments of the present invention;

FIG. 7 illustrates a dual swab stem handle for connecting two swab assemblies to make a dual swab assembly in accordance with certain preferred embodiments of the present invention;

FIG. 8 illustrates a dual swab assembly in accordance with certain preferred embodiments of the present invention:

FIGS. 9-10 illustrate overall device views of a fluid sample collection and testing device in accordance with additional certain preferred embodiments of the present invention;

FIGS. 11A and 11B illustrate a top cap preferably sonic weldable in accordance with additional certain preferred embodiments of the present invention:

FIG. 12 illustrates a dual swab stem handle for connecting two swab assemblies to make a dual swab assembly and a top cap therefor in accordance with additional certain preferred embodiments of the present invention;

FIG. 13 illustrates a lockable bottom cap for a fluid sample collection and testing device in accordance with additional certain preferred embodiments of the present invention;

FIGS. 14 and 15 illustrate a vial confirmation stem tube venting implement for a fluid sample collection and testing device in accordance with certain preferred embodiments of the present invention;

FIGS. 16A-16D illustrate certain floor properties and other attributes of a test chamber and a ventable/sealable gasket for a fluid sample collection and testing device in accordance with certain preferred embodiments of the present invention;

FIG. 17 illustrates an improved cassette for holding test strips for a fluid sample collection and testing device in accordance with certain preferred embodiments of the present invention;

FIGS. 18A-18B illustrate a ventable/sealable gasket for a fluid sample collection and testing device in accordance with certain preferred embodiments of the present invention;

FIG. 19 illustrates a swab assembly-housing holding clip for a fluid sample collection and testing device in accordance with certain preferred embodiments of the present invention;

FIG. 20 illustrates “Vial-Side Only” embodiments of the present invention, including use of indicator strips and/or lateral flow test strips in a swab stem;

FIG. 21A illustrates certain details of exemplary lateral flow test strip used in certain preferred embodiments of the present invention;

FIGS. 21B and 21C illustrate schematically single and dual lateral flow test strip used in certain preferred embodiments of the present invention;

FIG. 22 illustrates schematically an exemplary arrangement of back-to-back lateral flow test strip used in certain preferred embodiments of the present invention;

FIGS. 23A-23C illustrate exemplary alternative embodiments of lateral flow test strips integrably insertable into a swab stem from a top portion used in certain preferred embodiments of the present invention;

FIG. 23D illustrates an exemplary cap and swab stem top having retention and sealing attributes;

FIGS. 24A-24C illustrate exemplary alternative embodiments of lateral flow test strips integrably insertable into a swab stem from a top portion and having an opened volume at the swab end used in certain preferred embodiments of the present invention;

FIGS. 25A-26C illustrate exemplary embodiments of a strip holder and swab stem for positioning one or more test strips into the swab stem;

FIG. 27A-29D illustrate exemplary embodiments of improved movable vent gaskets/seals used in preferred embodiments of the present invention:

FIG. 30 illustrates an exemplary preferred embodiment of a Single Swab Device incorporating an improved movable vent gasket/seal;

FIG. 31 illustrates an exemplary preferred embodiment of a Single Swab Device incorporating an improved movable vent gasket/seal and/or a strip holder and swab stem for positioning one or more test strips into the swab stem;

FIGS. 32A-32B illustrates an exemplary preferred embodiment of a Dual Swab Device incorporating an improved movable vent gasket/seal and/or a strip holder and swab stem for positioning one or more test strips into the swab stem;

FIG. 33A-33B illustrates an exemplary preferred embodiment of a Single Swab Device incorporating a strip holder and swab stem for positioning one or more test strips into the swab stem, and

FIGS. 34 and 35 illustrate exemplary workflows and tests performed using an improved movable vent gasket/seal and/or a strip holder and swab stem for positioning one or more test strips into the swab stem.

### DETAILED DESCRIPTION OF THE PREFERRED EMBODIMENTS

The present invention will be described in greater detail with reference to certain preferred and alternative embodiments. As described below, refinements and substitutions of the various embodiments are possible based on the principles and teachings herein. It should be understood that this disclosure is directly primarily to: improved fluid collection systems and methods by which, for example, a fluid specimen may be collected via a swab and have a first portion of the collected specimen dispensed into an external vessel (such as a cassette or holder for testing) as a first step, and a second portion of the collected specimen dispenses into a specimen container as a second step; improved methods of compressing fluid specimens from a swab in which the swab is compressed by downward compression of the swab in a compression tube with a movable venting gasket/seal having one or more cross members impeding the swab but allowing passage of fluid specimen into an external test vessel, specimen container, or other location external to the compression tube, and/or improved swab assemblies having analyte test holders for holding one or more analyte test strips that may be inserted into a “top” end of the swab assembly that is distal from a “bottom” end of the swab assembly that has a sponge-like material for collecting a fluid specimen. It will be understood by those of skill in the art that the aforementioned improved aspects are combinable with the other implements and steps disclosed herein. For example, the improved movable venting gasket/seal may be used in any embodiment utilizing such a compression mechanism, and any swab assembly used herein may incorporate such an analyte test strip holder insertable into a “top” end of the swab assembly. All such refinements and combinations are expressly within the inventions claimed herein.

The following description will provide background, features, implements and processes usable in combination with the specific improvements disclosed herein.

As will be understood from the Referenced Patent Documents, particularly in view of this disclosure, the present invention utilizes a fluid (e.g., saliva) absorption or capture device (herein a “swab” or sponge/sponge-like material that may absorb/contain and release the fluid of interest) that preferably captures oral fluids such as by insertion of the swab into a subject's mouth, whereby oral fluids are captured/absorbed into the swab (where the term “saliva” is used, it is to be understood that other than oral fluids also may be used in accordance with embodiments of the present invention). As an illustrative example, in accordance with alternative embodiments of the present invention, a swab may be wetted with water or other fluid such as alcohol, then brought into contact with a dry, powder or other substance (which may be suspected to be a drug, contaminant or other material of interest or concern), and then swabs inserted into the device for testing. Each swab preferably is mounted on a stem type device so that the absorbent material of the swab/sponge may be readily positioned in the subject's mouth, with the stem holdable much like a lollipop. After a sufficient amount of time, e.g., 2-5 minutes, and preferably as indicated by an indicator implement (hereinafter described), saliva typically is captured in sufficient volume so as to be introduced via a dual swab into the housing of a Dual Swab Device for testing and preferably confirmation (as will be described in more detail hereinafter). In alternative Single Swab Devices, saliva typically is captured in sufficient volume so as to be introduced via a single swab into the housing of a Single Swab Device for testing (as will be described in more detail hereinafter). In both Single and Dual Swab Devices, insertion of the swab via the stem into the housing preferably causes compression of the swab into a bottom portion of a swab tube in the housing, forcing saliva to be released from the swab wherein pressure is generated such that saliva is forced or pushed from the swab tube into a lateral flow assay strip chamber through an opening sized for the particular fluid under examination (there is flow communication via a channel from the swab tube in the housing into strip chamber). The saliva contacts the strips whereby the previous of drugs, diseases or other substances may be detected in desirable levels (see the Referenced Patent Document). In Dual Swab Devices, a second swab tube is provided so that a second swab may be compressed such that saliva is released and captured into a vial, separate from the chamber described above housing the assay strips. Upon full insertion of the dual swabs, the unit is sealed so as to minimize fluid escape from the device. In Dual Swab Devices, a relatively prompt initial screening may be made via the assay strips, and a second sample is captured in the vial, which is separate from, and not in flow communication with, the chamber housing the strips, which may be made available for confirmation testing in a laboratory, such as in a high complexity via for example mass spectroscopy, such as Liquid Chromatography Mass Spectroscopy (LC-MS). In Single Swab Devices, the confirmation vial is not provided, and only the screening via the assay strips preferably is provided.

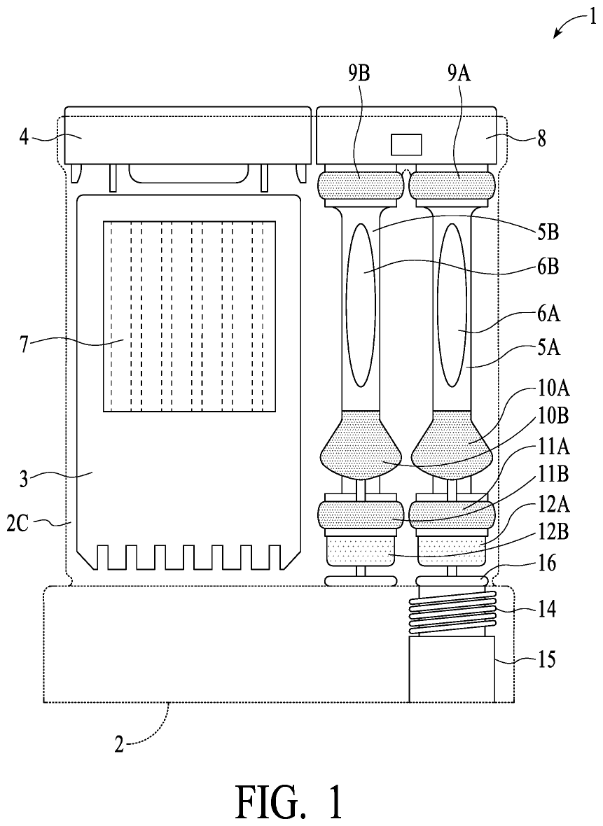

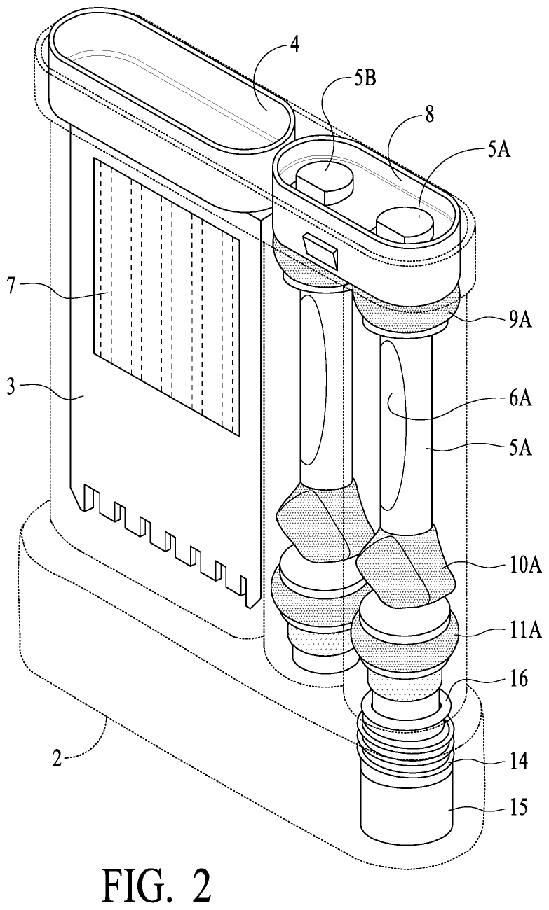

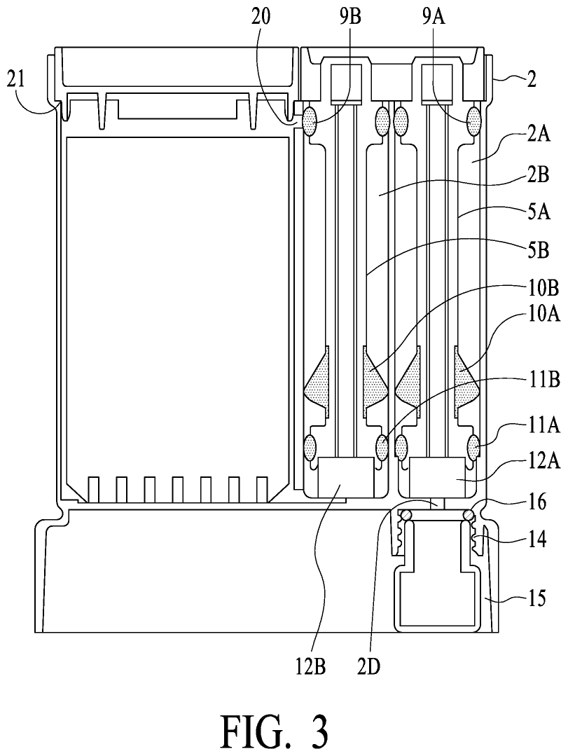

Referring now to FIGS. 1 and 2, an assembled Dual Swab Device 1 is illustrated. Housing 2 (preferably plastic such poly carbonate PC or general purpose polystyrene GPPS or similar materials) provide the main body of the unit. In a test chamber as illustrated, strip card/carrier or cassette 3 is provided, which includes one or a plurality of assay strips generally illustrated as 7. In operation, fluids are introduced into test chamber 2C of housing 2 so that the presence or absence of one or more target analytes (e.g., drugs, disease markers, etc.) may be detected based on the particular characteristics of the one or more strips 7. Test chamber 2C of housing 2 has an upper body plug or top cap 4, which preferably is sonic welding to housing 2 to provide an air and water tight seal, with the sonic welding carried out after cassette 3 has been inserted into test chamber 2C. As illustrated, a Dual Swab Device preferably includes two swab assemblies, connected via a preferably snap together, locking cap or dual stem handle 8 (each swab assembly is inserted into stem handle 8 and each has a locking feature for engaging a corresponding feature in stem handle 8 to lock them together to create a dual swab assembly). Each of swab assemblies 5A and 5B consist generally of the following: stems 6A and 6B (preferably plastics such as described for housing 2) having at least a visual window (indicated by ovals in FIG. 1) such that color changes of an indicator strip inside of stems 6A and 6B may be visible to an observer (and possibly the subject) (indicator strips are described in greater detail hereinafter, and such description is applicable here); preferably soft or elastomeric type material (such as by “second shotting” or “over molding” provide seals 9A and 9B at an upper portion of the swab assemblies, and seals 11A and 11B at a lower portion of the swab assemblies; swabs 12A and 12B (absorbent or sponge like material, such as described elsewhere herein), preferably secured by adhesive or other attachment, are secured to the bottom of stems 6A and 6B and provide the saliva capture for the devices (shown in compressed form in FIG. 1); tooth rests 10A and 10B, preferably of a similar material as seals 9A, 9B, 11A and 11B, are preferably provided so that a comfortable and desired resting place for front teeth of the subject (this enables the swab stem to be more comfortably held in a manner so that swabs 12A and 12B may more optimally capture oral fluids, etc.). Vial 15 is provided in Dual Swab Devices so that a confirmation sample may be obtained. Vial 15 preferably is secured to housing 2 via threads 14 of housing 2 or similar securing mechanism, and preferably sealed with O ring 16. As should be understood based on the description herein, swab assembly 5A may be compressed in tube 2A of housing 2 (see FIG. 3) and release oral fluid into vial 15 via fluid channel 2D (vial 15 is in fluid communication with tube 2A as illustrated), and swab assembly 5B may be compressed in tube 2B of housing 2 and release oral fluid into test chamber 2C via channel 23 (see FIG. 4) (test chamber 2C is in fluid communication with tube 2B via channel 23 as illustrated).

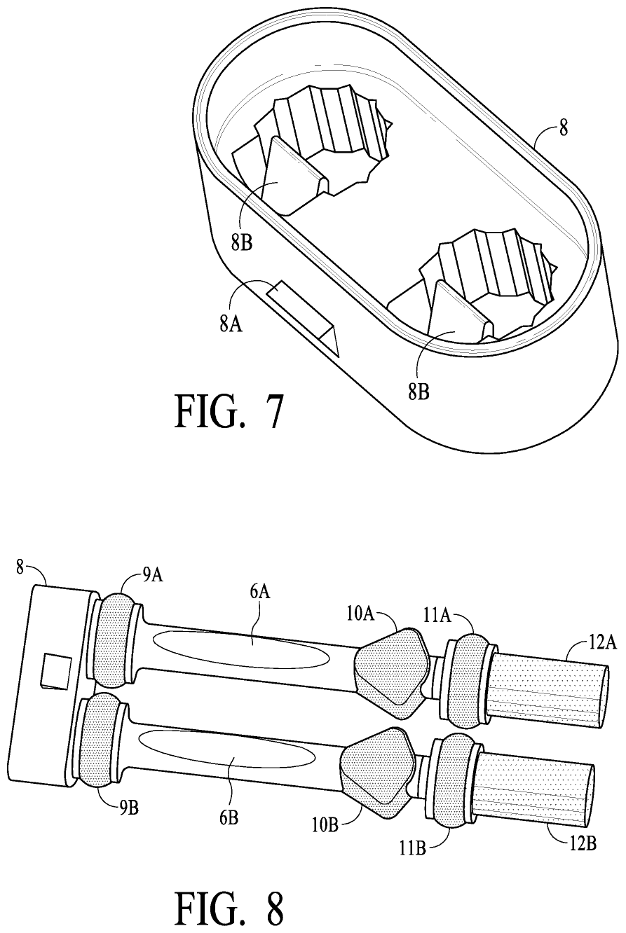

FIG. 3 illustrates additional attributes of preferred embodiments of the present invention, in cross section. Focusing now on the left most swab assembly 5B and swab tube 2B of housing 2 (herein sometimes called the “strip swab tube”), strip swab assembly 5B is shown in cross section fully inserted into the strip swab tube (swab 12B is compressed). Swab tube 2B preferably has a diameter of a first dimension, and seal 11B has a dimension such as to form a relatively tight fit/seal with swab tube 2B. As strip swab assembly 5B is inserted into swab tube 2B, the relatively tight fit/seal causes compression of swab 12B (and saliva release from the swab) and results in interior pressure in the lower portion of the swab tube 2B, with the result that saliva is released from swab 12B and forced or pressured moved into test chamber 2C of housing 2 via an opening (see opening or saliva passage 23 of FIG. 4). The size of this opening preferably is determined based on, for example, the particular fluid being tested and its characteristics, such as viscosity. What is important is that the relatively tight fit/seal of swab assembly 5B and the diameters of swab tube 2B and seal 11B are such that a plunger/compressive effect is achieved, and saliva is released and forced via opening/passage 23 into test chamber 2C. As interior pressure in test chamber 2C of housing 2 can increase, in preferred embodiment air is vented from test chamber 2C into test swab tube 2B via a vent opening (see opening/vent 20 of FIG. 3, which provides flow communication for air venting between test chamber 2C and swab tube 2B). Note that there is not a seal present at the top of swab tube 2B while swab assembly 5B is being inserted (stem 6B of swab assembly 5B being less in cross section than the interior dimension of swab tube 2B). Thus, while being inserted, pressure from the compressed swab forces saliva into test chamber 2C, air is vented from test chamber 2C via opening/vent 20 into swab tube 2B where it may escape out the top, thereby reducing “back pressure” experienced by the user pushing swab assemblies 5A and 5B into housing 2. Once swab assembly 5B, however, is fully inserted into swab tube 2B (and preferably locked such as with a locking tab—see FIG. 7 and dual stem handle 8 and locking tab 8A, which will be understood to engage with an indentation or hole of an upper portion of the tube section of housing 2 to provide a locking mechanism), seal 9B seals swab assembly 5B against swab tube 2B AND seals off opening/vent 20. Thus, operationally, opening/vent 20 allows air release during the insertion process (to prevent pressure build-up in the test chamber as saliva is forced in), and then is closed by a preferably overmold seal upon full insertion of the swab tube assembly.

The right most swab tube 2A of housing 2 (herein sometimes referred to as “confirmation swab tube”) operationally receives swab assembly 5A. Swab tube 5A has a second dimension (illustrated as 2E preferably down to a lower extremity as illustrated), and seal 11A has a diameter such as to form a relatively loose fit/seal with swab tube 5A (and is not air tight but substantially water tight). Thus, upon insertion of swab assembly 5A into swab tube 2A, swab 12A is compressed and saliva is released into vial 15 via fluid channel 2D, while air may be vented out via the top of swab tube 2A around lower seal 11A. Swab tube 2A preferably has a shoulder (narrower diameter of a third dimension, less than the second dimension, generally illustrated as 2F) at a lower portion such that seal 11A may tightly engage the shoulder (2F) and seal vial 15 from other portions of the unit upon swab assembly 5A being fully inserted into swab tube 2A (see seal 11A in FIG. 4, showing the engagement of seal 11A with the shoulder (2F) of swab tube 2A). FIG. 4 also illustrates a seal between vial 15 and housing 2 (see o-ring seal 16 of FIG. 4), and securing of vial 15 to housing 2 such as by a thread mechanism (see vial threads 26 of FIG. 4, which engage corresponding threads 14 of housing 2). What is important is that, while swab assemblies 5A and 5B preferably are of the same size, the diameters of swab tube 2B and swab tube 2A are of different dimensions, such that pressure is generated to force saliva into test chamber 2D (with air vented from test chamber 2D via opening/vent 20 to swab tube 2B), and air from confirmation swab tube 2A is vented from the top of confirmation swab tube 2A. In exemplary preferred embodiments, the third dimension is less than the first and second dimensions.

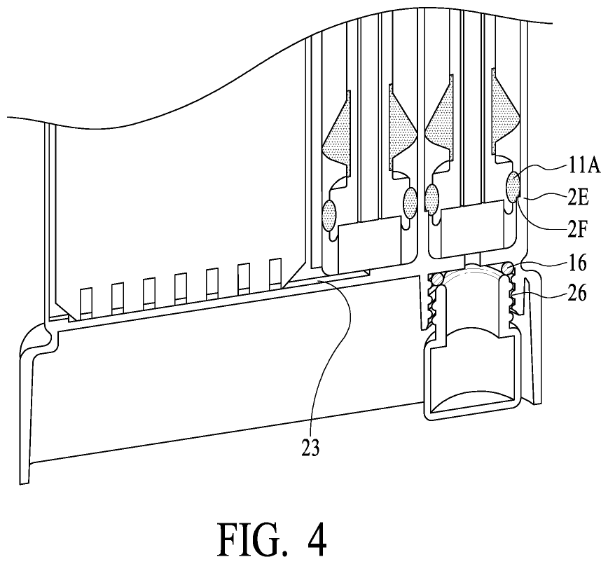

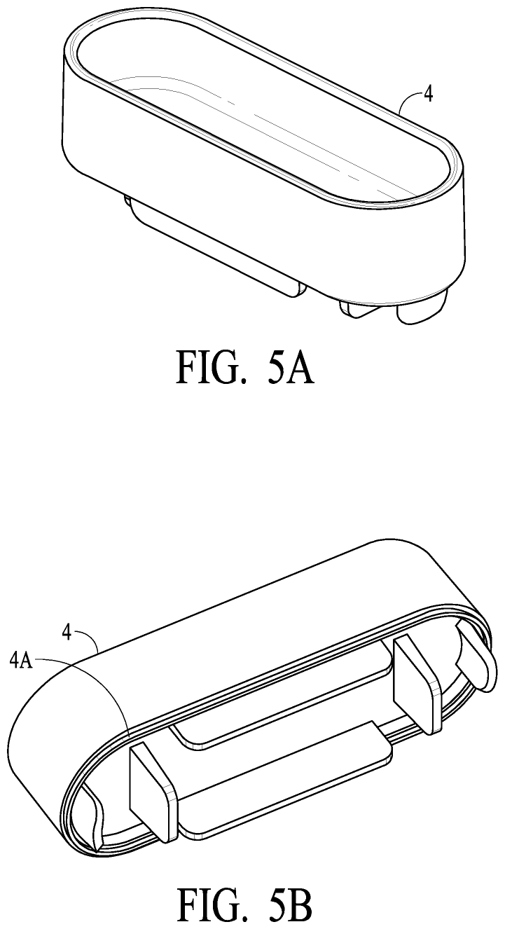

FIGS. 5A and 5B illustrate body top cap 4, which is sealed to the top of the test chamber of housing 2 preferably via sonic weld to create a air and water tight seal. Body top cap 4 preferably has energy director 4A such as to facilitate the sonic weld process.

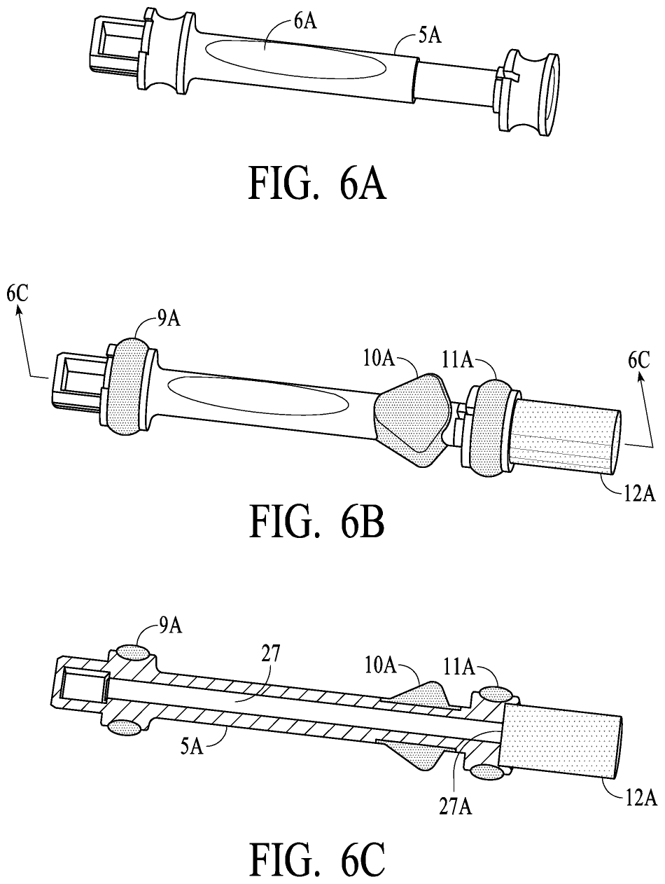

FIGS. 6A-6C illustrate swab assembly 5A is various stages of manufacture. FIG. 6A will be understood to be after the first shot, or first injection molding, of the plastic of swab assembly 5A (stem 6A is illustrated). FIG. 6B will be understood to be after the second shot, or second injection molding of the softer, more compliant material that is used, preferably, for seals 11A and 11B and teeth rests 10A and 10B as described elsewhere herein. FIG. 6C illustrates swab assembly 5A in cross section such that indicator strip 27 is visible. In preferred embodiments, swab assembly 5A (and preferably 5B) has interior portion with an opening (preferably a slit) such that an indicator strip 27 may be positioned to engage with swab 12A at one end (illustrated as contact point 27A), and then extend up stem 6A so as to be visible through the oval window of stem 6A. As will be understood, indicator strip 27 changes color when wet or saturated, and is calibrated/configures to serve as an indicator that a substantial and preferably sufficient amount of saliva has been captured by swabs 12A and 12B so that upon insertion of the swab assemblies (1) strips 7 are activated to provide a quick screening for target substances/analytes, etc., and saliva is captured in vial 15 for subsequent laboratory/confirmation testing if needed or desired. Preferably, indicator strip 27 is visible to an observer and/or subject and provides a confidence indicator that sufficient saliva has been captured prior to insertion of swab assemblies 5A and 5B into housing 2.

As will be understood, the present invention encompasses both Single and Dual Swab Devices. In a Single Swab Device, swab tube 2A, swab assembly 5A, vial 15, etc. are not included, and thus housing 2 may be smaller (and not, for example, include the lower skirt portion that surround and protects vial 15. In preferred embodiments, however, both Single and Dual Swab Devices are provided with a common swab assembly (such that robotic equipment may be commonly used for production of both Single and Dual Swab Devices), and a dual swab assembly is produced by securing two single swab assemblies 5A and 5 with dual stem handle 8 (see, e.g., FIG. 7). Having a single swab assembly commonly used for Single and Dual Swab Devices with a locking swab step handle (such as with locking tab 8B, which locks into a corresponding feature of swab stems 6A and 6B, such as illustrated and/or described elsewhere herein) provides significant advantages in assembly cost and time. FIG. 8 is a view of a dual swab assembly using dual stem handle, shown an exemplary locking tab for securing permanently (absent breakage) two single swab assemblies into a dual swab assembly. As will be understood, stem handle 8 is adapted to having an opening and locking mechanism to secure only one swab assembly in Single Swab Device embodiments, as will be understood by one of skill in the art.

The present invention may utilize a wide variety of strips as desired for the particular application. In one additional attribute, a strip testing for THC (parent or metabolite) is positioned in proximity to the opening to swab tube 2B so that saliva may be initially forced onto the THC sensing strip. Increased efficiency of THC testing is an important attribute of the present invention. See other description elsewhere for additional exemplary strips and analytes, etc.

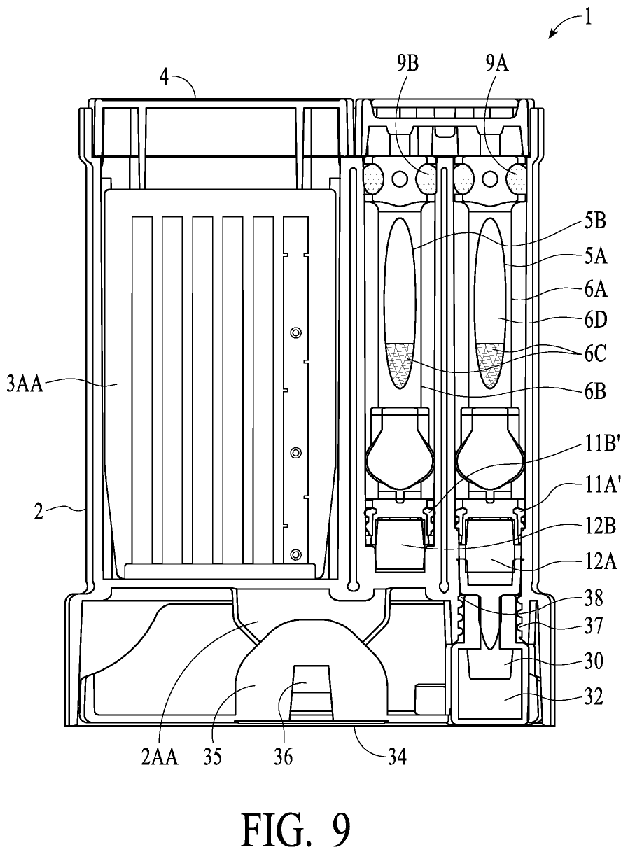

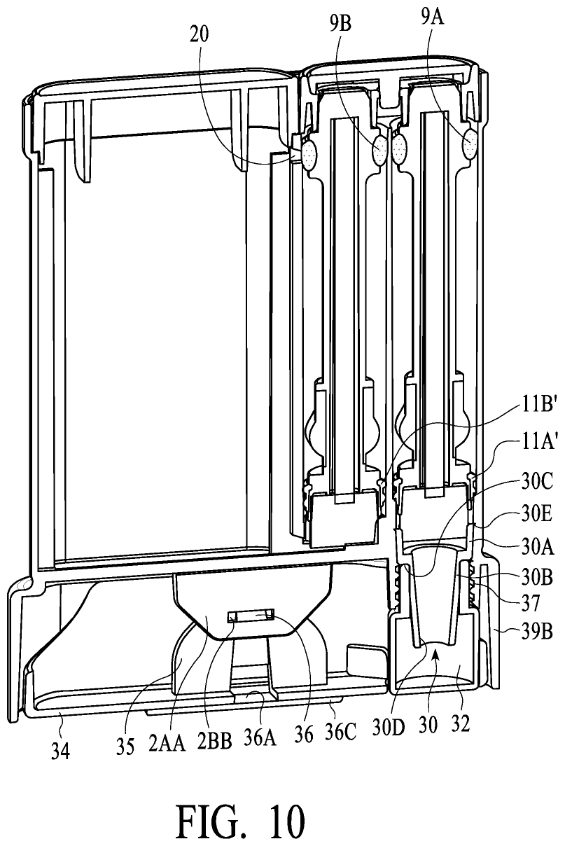

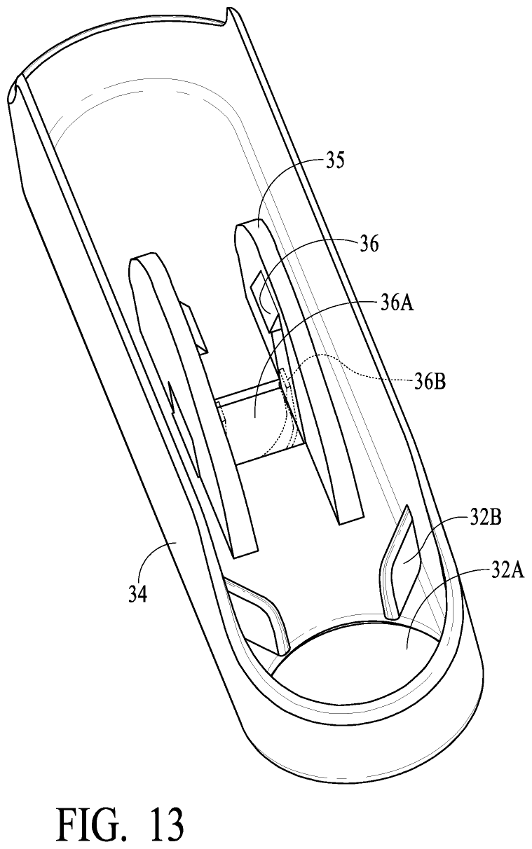

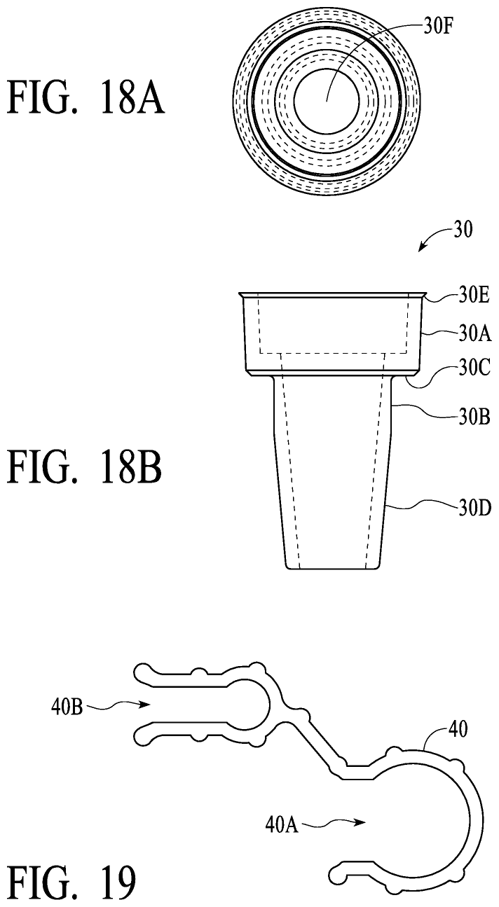

Referring now to FIGS. 9-19, additional exemplary preferred embodiments will be described. As illustrated in FIG. 9, housing 2 in alternative preferred embodiments include interior tabs 2AA near the bottom of housing 2. Tabs 2AA extends downward in a bottom cavity portion of housing 2 as also illustrated in FIG. 10, and includes openings 2BB. Such preferred embodiments preferably include bottom cap 34, which includes tabs 35 with projections 36 (in certain preferred embodiments two of such projections 36 are provided), and projections 36 engage with openings 2BB such that bottom cap 34 preferably is locked onto housing 2. In such preferred embodiments, bottom cap 34 includes key opening 36A, into which a key or tool (illustrated by dotted lines 36B in FIG. 13) may be inserted to cause projections 36 to disengage from openings 2BB such that bottom cap 34 may be removed from housing 2. In certain preferred embodiments, tape 36C (which may be an adhesive or other blocking material that is puncturable) covers opening 36A, thereby providing evidence of tampering or lack thereof. In operation, tool 36B (or other protrusion) punctures tape 36C in order to engage projections 36 (in order to remove bottom cap 34). In this manner, vial 32 (similar to vial 15, described elsewhere herein) may be accessed for removal, such as for having a laboratory confirmation of the preferably oral fluid sample there preferably using an LC MS machine, by puncture of tape 36C and removal of bottom cap 34. Intact tape 36C is evidence that the unit has not been opened or tampered with.

Additional details of bottom cap 34 are shown for illustrative purposes in FIG. 13, which illustrates tool 36B (shown as dotted lines in FIG. 13 to illustrate a member that is inserted into opening 36A to spread projections 36 to facilitate removal of bottom cap 34) engaging tabs 35 to cause movement of projections 36 (so as to disengage from openings 2BB). Tool 36B is illustrated to have two parallel or substantially parallel projecting members (shaped much like a flat head screwdriver tip) that engage with tabs 35. The precise shape may vary; what is important is that tool 36B be projectable through hole 36A and cause movement of projections 36 to enough bottom cap 34 to be removed via the tool. Bottom cap 34 preferably (but optionally) includes opening 32A into which the bottom end of vial 32 extends, facilitating viewing of vial 32 from the bottom, etc. Ribs or members 32B are provided to engage vial 32 and help ensure that vial stays securely in housing 2 (and is not, for example, loosened such as by vibration during transport of the device). Contact between ribs/members 32B put pressure on vial 32, causing vial 32 to resist rotational unthreading from housing 2. Vial 32 is secured to housing 2 via threads 37 (similar to threads described elsewhere herein).

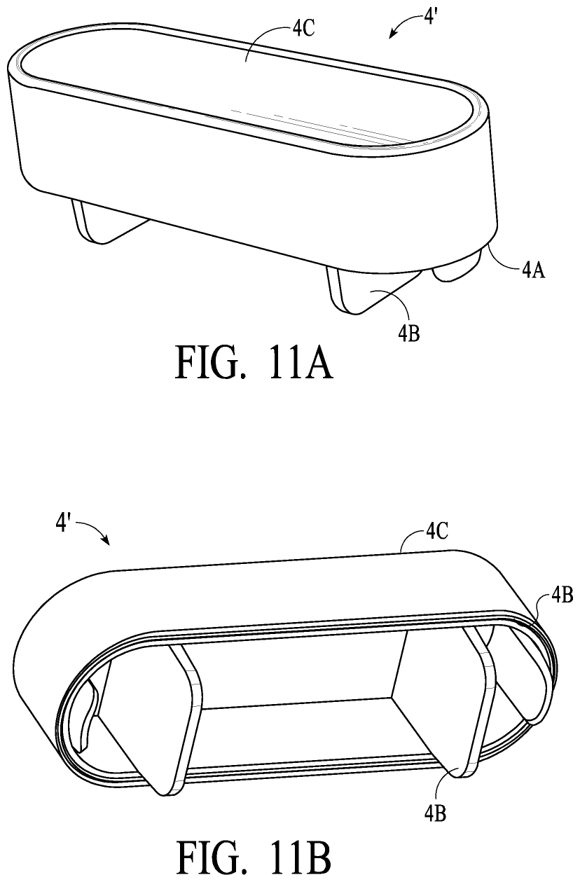

Referring again to FIG. 9 and also FIGS. 11A and 11B, alternative upper body top cap 4′ is illustrated. Top cap 4 preferably has a flat top 4C as illustrated and preferably includes tabs 4B extends downward to engage strip card/carrier (or cassette) 3, helping to hold cassette 3 in place so that the bottom tips of the strips carried by cassette 3 are positioned in proximity to the bottom of test chamber 2C of housing 2 so as to contact oral fluid when the device is in operation. Top cap 4′ preferably includes sonic welding energy director surface 4A, which engages housing 2 so that it may be adhered to housing 2 preferably by sonic welding. While adhesives or other securing elements may be used to secure top cap 4′ to housing 2, it has been determined that sonic welding preferably provides a low cost and air and water tight secure securing mechanism, also providing tamper resistance to the strips within test chamber 2C (venting is provided by opening 20, sealed by seal 11B, such as described elsewhere herein).

Referring again to FIG. 9, swab assemblies 5A and 5B are illustrated in a downward and locked position. Also as illustrated, each of swab stems 6A and 6B include saliva indicator strips 6D (similar to indicator strip 27 described elsewhere herein), each of which includes a color changing region 6C, which preferably changes (for example) from a first color (e.g., white) to a second color (e.g., red) when oral fluid wicks up from the swab absorbent tip (swabs 12A and 12B) onto strips 6D in swab stems 6A and 6B. In preferred embodiments, indicator strips 6D enable an indication of adequate oral fluid being absorbed in swabs or sponges 12A and 12B (see also FIGS. 1-6C) such that oral fluid will contact test strips in cassette 3 and occupy a substantial volume of vial 32 when swab assemblies 5A and 5B are inserted and locked into housing 2. As illustrated, indicator strips 6D (or 27) may have color changing region 6C on one of the indicator strip, and in preferred embodiments color changing regions 6C on both swabs of a Dual Swab Device face commonly in an up or down direction as illustrated.

Referring again to FIGS. 9 and 10, further sealing attributes of the additional exemplary preferred embodiments will be described. As described previously, seals 9A. 9B, were provided at the upper portion of swab stems 6A and 6B, which will be understood to provide a seal to the outside from the interior of both swab tubes 2A and 2B by pressure contact with the walls of swab tubes 2A and 2B by seals 9A and 9B. Seal 9B serves the additional purpose of sealing opening/vent 20 when swab assemblies 5A and 5B are in a fully inserted and preferably locked position in housing 2. As will be appreciated from the drawings and the description herein, Seals 9A and 9B are adapted to form a type of stopper seal, or a rounded contour seal, for opening/vent 20 and the tops of swab tubes 2A and 2B. In the previously described exemplary preferred embodiments, lower seals 11A and 11B are formed similarly to seals 9A and 9B. In alternate exemplary preferred embodiments, sealed 11A and 11B are formed in a different manner.

FIGS. 9 and 10 illustrate lower seals 11A′ and 11B′ (shown in cross section) having a shape adapted to form a sliding seal (rather than a stopper seal). Applicants have determined that in certain embodiments having a sliding type or syringe type lower seal enables swab assembly insertion into the swab tubes with reduced friction or resistance. As illustrated in such embodiments, seals 11A′ and 11B′ preferably are formed to have a contoured or textured profile, as compared to the rounded contour of seals 9A and 9B. In one illustrative example, seals 11A′ and 11B′ have one or more preferably a plurality (two being shown as an example) fins or o-rings (projecting sealing members, etc.), preferably spaced apart as illustrated but formed in a unitary manner, such as by a second shot or overmold injection molding process. Such fins or o-rings provided a sliding seal or a syringe seal to prevent or inhibit fluid travel up as the swab assemblies 5A and 5B are inserted and moved downwardly in swab tubes 2A and 2B. As swabs 12A and 12B are compressed by downward movement of swab assemblies 5A and 5B into swab tubes 2A and 2B and fluid is released from swabs 12A and 12B, pressure builds, and sealing is provided by seals 11A′ and 11B′ to inhibit upward flow of the fluid towards the top of the housing, thereby facilitating fluid flow into vial 32 and test chamber 2C. At the same time, user experience degrades if the force required to fully insert swab assemblies 5A and 5B is too great, and thus, swab seals 11A′ and 11B′ are provided in a form that provides an adequate seal with improved slide resistance by having a contoured or finned structure or textured shape. In yet further alternative embodiments, seals 11A′ and 11B′ are textured in a manner to reduce sliding resistance rather than with projection-style fins or o-rings in a manner to provide a sliding/syringe type seal, again as compared to the stopper type seal of seals 9A and 9B. What is important is that each of seals 9A/9B and 11A′/11B′ are adapted and formed optimally for their particular functions, which is understood from the description and drawings provided herein, and preferably formed from the same manufacturing steps, such as a second shot or overmold injection molding process that form both types of seals on swab stems 6A and 6B.

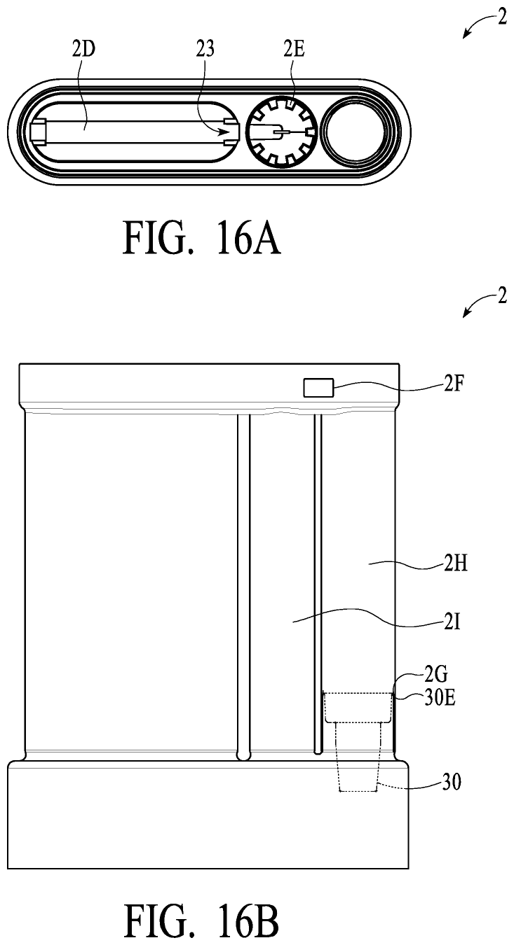

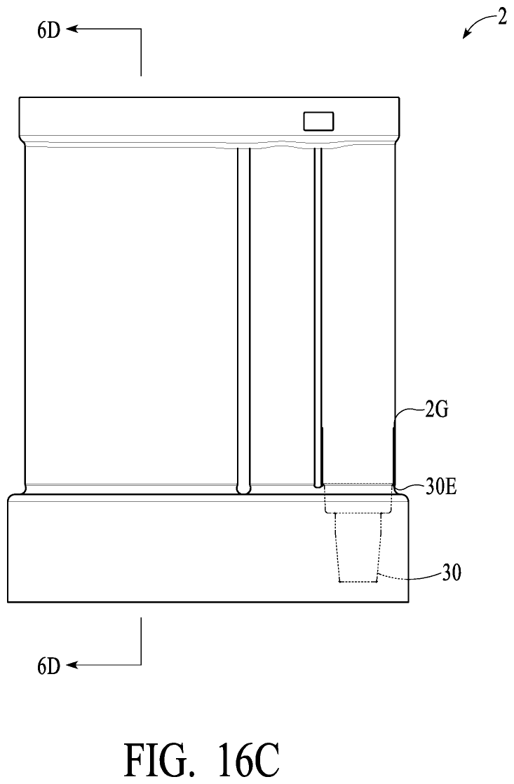

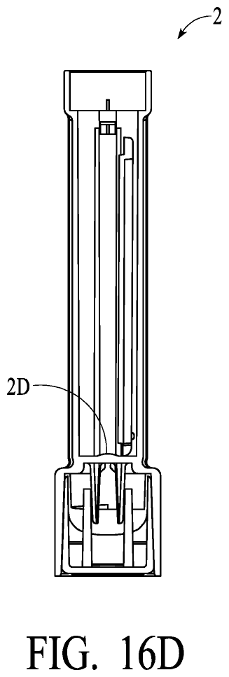

The additional exemplary preferred embodiments illustrated in FIGS. 9-19 also include a vented thread/gasket sealing mechanism, which is further illustrated and described herein. FIGS. 9, 10, 16A-16D, 18A and 18B illustrate gasket 30, preferably shaped much like a funnel, and preferably having extending portion 30D that extends into vial 32 and channels a flow of fluid (which may be oral fluid) into vial 32 as swab 12A is compressed upon insertion into housing 2. Gasket 30 has opening 30F through which fluid passes into vial 32. In a first position illustrated at the height shown by gasket 30 in FIG. 16B, upper portion 30A of gasket is above the top of vial 32 resting on ledge 2G of housing 2, with ledge 2G being a slightly reduced diameter portion of the vial-side housing tube (2H in FIG. 16B, similar to swab tube 2A described elsewhere herein). Lip 30E of gasket 30 rests on ledge 2G, and friction interference between upper portion 30A and the wall of vial side housing tube 2H create a seal inhibiting passage of oral fluid. In this first position, as swabs/sponges 12A and 12B are compressed as swab assembly 5A is inserted and lowered into housing 2, oral fluid is released from swab/sponges 12A and 12B and flows into vial 32 via opening 32F (and test chamber 2C as previously described via a flow communication channel as previously described). Swabs/sponges 12A and 12B are further compressed into the downward pressure position, as illustrated in FIGS. 9 and 10. In the second position, as illustrated in FIG. 16C, gasket 30 preferably has upper portion 30A contacting the side circumferential wall at the lower end of vial tube 2H, under surface 30C downwardly contacting the top circumferential surface of vial 32, and neck surface 30B contacting the inner circumferential wall of the neck portion of vial 32 (on the outside of which via 32 has threads 37 engaging corresponding threads of housing 2 to secure vial 32 to housing 2).

In accordance with preferred embodiments, gasket 30 is formed of a flexible, but relatively rigid, preferably elastomeric material such as TPE (thermoplastic elastomer) or other polymeric or rubber like material. In accordance with preferred embodiments, gasket 30 has a durometer of greater than 50, and more preferably a durometer rating in the range of 70 to 90. In accordance with certain preferred embodiments, gasket 30 is a flexible material having a durometer of 80+/−5-10%. Applicant has discovered that such a composition of gasket 30 desirably allows gasket 30 to hold in the first position while having enough compression to inhibit oral fluid from flowing around gasket 30 while in the first position, but yet release, as pressure increases during downward movement of the swab assemblies 5A and 5B, at a reasonable pressure to move and traverse down swab tube 2H to the second position, thereby bringing surfaces 30B and 30 C into contact with the corresponding surfaces of vial 32, and bringing surface 30A into contact with the circumferential wall of vial tube 2H of housing 2 so as to inhibit oral fluid from flowing around gasket 30 while in the second position.

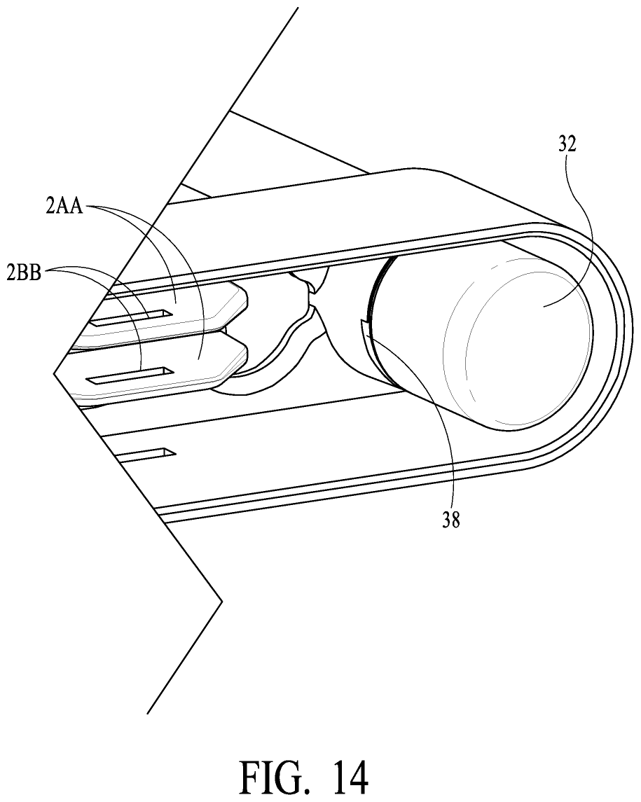

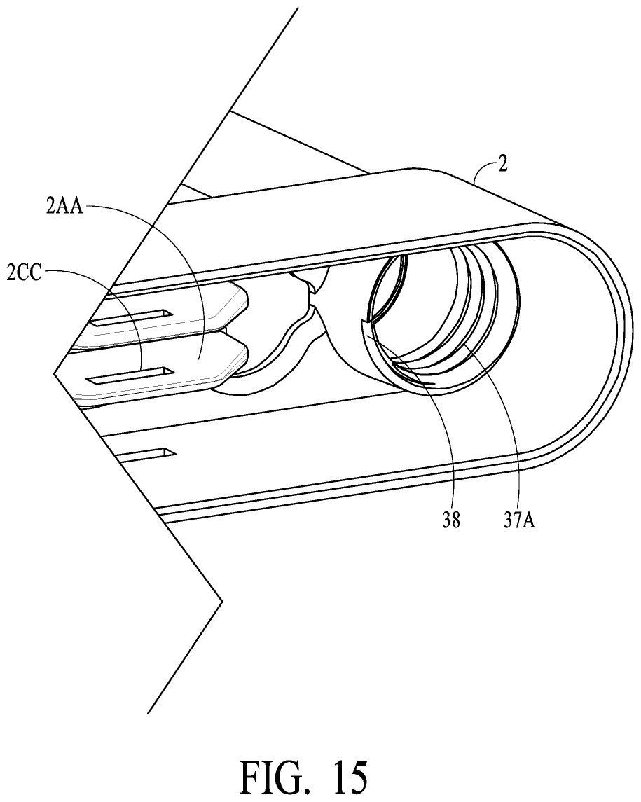

Having gasket 30, housing 2 and vial 32 configured and arranged as illustrated and described herein, certain preferred embodiments utilize improved air venting so as to provide reduced back pressure to a user attempting to push swab assemblies 5A and 5B into locked position in housing 2, and improved fluid extraction efficiency. As illustrated in FIGS. 14 and 15, notch 38 is formed in the lower end portion of threaded wall 37A of housing 2. Notch 38, which may be tapered or contoured, ensures a gap between at least part of the lower end portion of threaded wall 37A of housing 2 (an arrow indicating air flow may be drawn from inside the vial around lower tip 30D and up the neck of vial 32 and around the threads and out notch 38). As vial 32 is secured onto housing 2 using threads 37 of vial 32 and threaded wall 37A of housing 2, the upper lip of vial 32 may contact housing 2 and snug-up to be secure, while notch 38 ensures that there is not an air-tight seal created at this contact point. Notch 38 thus enables air venting via threads 37/threading wall 37A. In accordance with such embodiments, sealing to prevent leaking of oral fluid is achieved via gasket 30 as previously described. Thus, as swabs/sponges 12A and 12B compress, air is vented via notch 38 and threads 37/threaded wall 37A, while fluid flow into vial 32 is facilitated, and an operator experiences less back pressure while pushing the overall swab assembly into its final locked position in housing 2. As will be appreciated, gasket 30 facilitates a linearly movable, two position valve/sealing implement or member, wherein in a first (unsealed, venting) position in swab tube 2A, air vents to the outside of the device, and under pressure of swab compression the gasket linearly moves/traverses down swab tube 2H to a second (sealed/non-venting) position in swab 2B, where air does not vent to the outside of the device, and fluid likewise does not escape to the outside of the device.

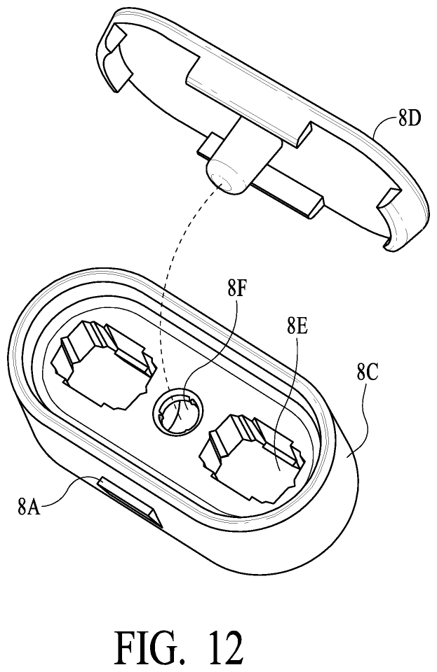

As illustrated in FIGS. 12 and 16B and 16C, housing 2 preferably includes openings 2F into which protrusions 8A on both sides of swab top collar 8C engage to lock the overall swab assemblies into housing 2. As illustrated, protrusions 8A preferably are tapered with an upper ledge to lock into openings 2F of housing 2, while in other embodiments other shapes of protrusions may be utilized. What is important is that the overall swab assembly lock into housing 2 so as to dissuade or prevent swab assembly removal and prevent/inhibit tampering. Also as illustrated, openings 8E are provided to engage with swab stems preferably having a similar protrusion to lock swab stems into place and an opening 8F to ensure a corresponding feature of swab assemble top cap 8D, such as is illustrated in FIG. 12.

Also as illustrated in FIGS. 16A and 16D, housing 2 preferably has a raised (preferably tapered or contoured) center portion 2D on the floor of housing 2 where the lower end of cassette(s) 3AA (FIG. 9) rest(s). Such a raised center portion floor helps channel fluid from cassette tube 2I of housing 2 to the lower ends of the strips inserted in cassette(s) 3AA by using gravity to flow oral fluid from the center region of test chamber 2C towards its edges where the strip tips preferably are positioned, thereby facilitating testing of the fluid via the strips. Housing 2 of FIGS. 9 and 10 includes passage/channel 23 from tube 21 (similar to swab tube 2B described elsewhere herein) into test chamber 2C of housing 2 containing cassette 3AA and an upper air opening/vent 20 such as previously described in connection with other preferred embodiments, Also, as illustrated in FIG. 16A, the floor of tube 2I preferably includes a plurality of raised bumps 2E to help compress swab/sponge 12B to facilitate release of oral fluid from swab/sponge 12B and movement to passage/channel 23 from tube 2I into the cavity containing cassette 3AA. See FIGS. 3 and 4 and related description regarding passage/channel 23 and opening/vent 20.

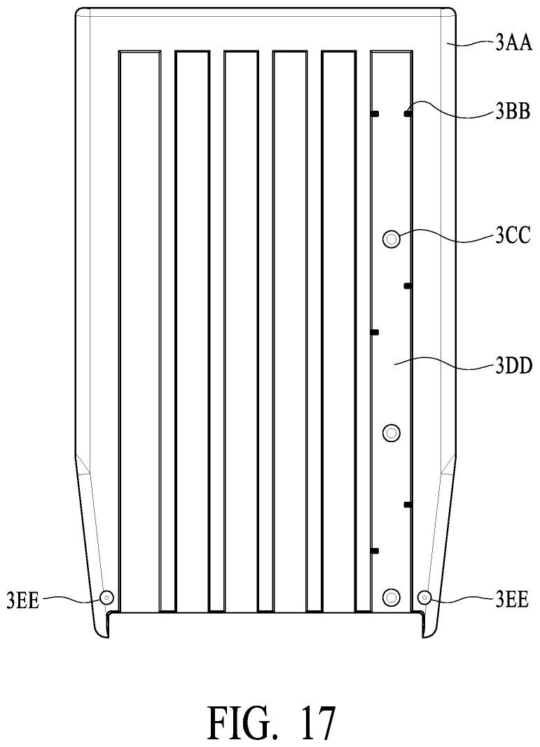

FIG. 17 illustrates an alternative preferred embodiment of a cassette for holding test strips having one or more channels, generally illustrated and described herein as cassette 3AA. It should be understood that the present invention contemplates embodiments including one or two such cassettes 3AA (or similar cassettes) on one or both sides of housing 2 as will be understood by those of skill in the art. As illustrated in FIG. 17, rightmost channel 3DD (a channel being a receptacle or holding area for a test strip) includes bumps or islands 3CC (three such bumps or islands 3CC shown in this exemplary embodiment) that causes the strip to be raised from the bottom floor of channel 3DD. In this manner, the backside of the strip has a spacing from (and thus reduced contact with) the material (preferably plastic) comprising cassette 3AA. In addition, channel 3DD includes side protrusions 3BB to position the strip so that the sides of the strip do not contact side walls of channel 3DD. Side protrusions 3BB preferably are on opposite sides of channel 3DD but preferably not directly across from each other as illustrated (in other embodiments, side protrusions 3BB are directly opposite each other). In this manner, the side edges of the strip have a spacing from and do not contact the side walls of the material comprising cassette 3AA. With the combination of bumps/islands 3CC and side protrusions 3BB, the strip more nearly floats in channel 3DD with reduced contact with the material comprising cassette 3AA. As will be understood by those of skill in the art, a label generally is provided to cover the strips as positioned in cassette 3AA. Applicants have determined that cassette 3AA having one or more (including up to all) channels of cassette 3AA constructed as shown provide improved detection of substances such as THC. In accordance with certain preferred embodiments, the strip for detecting the presence or absence of THC is positioned in a channel such as channel 3DD positioned on a rightmost or leftmost channel of cassette 3AA (having a plurality of channels, such as the 6 channels shown in FIG. 17 or the 7 channels illustrated in FIGS. 1 and 2) so as to more optimally contact oral fluid. In alternative preferred embodiments, all such channels of cassette 3AA have bumps/islands 3CC and side protrusions 3BB as is shown for channel 3DD of FIG. 17.

In preferred embodiments, cassette 3AA also includes bumps 3EE, which serve to position cassette 3AA away from front face of housing 2, which has been determined to reduce capillary rise of fluid upwards between cassette 3AA and housing 2. In this embodiment, bumps 3EE may contact the front face of housing 2, while the main face of cassette 3AA is spaced apart from, and does not contact, the front face of housing 2. Additionally, as illustrated cassette 3AA may optionally have side legs having a tapered or beveled shape, so as to have a smaller dimension at the bottom of the legs and a larger dimension at an upward portion of the legs. In this manner, the smaller dimension is presented to fluid at the bottom of test chamber 2C, further reducing the incidence of capillary rise or fluid contact with the material of cassette 3AA.

As illustrated in FIG. 19, holding clip 40 is provided so that a complete swab assembly such as shown in FIG. 8 may be secured by clamp 40B, while clamp 40A clamps to vial tube 2H. In this manner, the complete swab assembly is secured to the outside of housing 2 so that the swab assembly/housing combination may be run through an automated pouching machine. In accordance with such alternative preferred embodiments, the swab assembly may be packaged in a plastic bag for hygienic considerations, and yet secured to housing 2 in a manner suitable for automatic pouching.

Applicants' exemplary embodiments expressly include the Single Swab Device, adapted to leave off the confirmation vial side of the device, including the swab tube and swab assembly in fluid communication with the confirmation vial. Thus, attributes of housing 2, test chamber 2C, opening/vent 20, channel 23, seals 9B, 11B, 11B′, etc., are desirably utilized with the Single Swab Device, as well as cassette 3AA and its variations as described and shown herein.

In accordance with yet other embodiments of the present invention, a Vial-Side Only Device is contemplated, which in essence may incorporate only the vial side of the device, including the swab tube and swab assembly in fluid communication with the confirmation vial. Thus, the description related to the vial side of the device is applicable to such Vial-Side Only embodiments and is hereby incorporated by reference. As will be understood by those of skill in the art, the plastic molding and component fabrication would be adapted to produce only the vial side of the device, with, for example, a top and bottom cap each adapted for just the vial side, using only a single swab (which preferably may be implemented in a manner consistent with the disclosed embodiments of the swab assemblies for the Dual Swab Device).

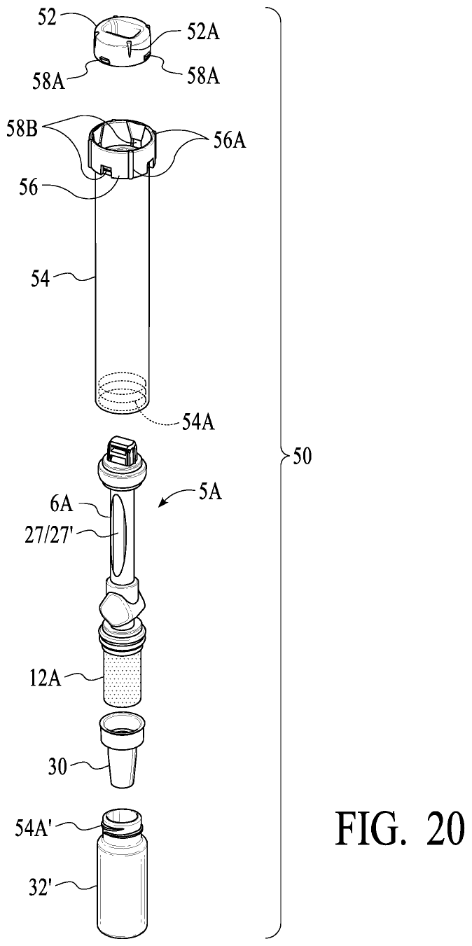

Refinements for such a Vial-Side Only Device are used in accordance with other exemplary preferred embodiments, one such embodiment being illustrated in FIG. 20, which is an exploded view of such embodiment. As illustrated, top cap 52 snaps onto the top of swab assembly 5A, which will thereafter be pushed into tube 54 when the device is run by a user (see explanation elsewhere herein). Top cap 52 includes tabs 58A having an upper ledge and tapered bottom to engage openings 58B of collar 56 of tube 54 when swab assembly 5A is “snapped” into final position. Preferably, the engagement of tabs 58A and openings 58B produces an audible snap sound, which (1) ensures that swab assembly 5A is fully inserted into tube 54, and (2) locks swab assembly 5A in position so that back pressure or inadvertent user operation does not cause swab assembly 5A to undesirably back out of tube 54. Collar 56 has slanted portions 56A of a larger dimension at the top to guide tabs 58A to engage with openings 58B. To aid in alignment of top cap 52 and tube 54 when a test is run, alignment marks 52A and 56A are provided on top cap 52 and collar 56 of tube 54 as illustrated. Four pairs of tabs 58A and openings 58B are in the illustrated embodiment, but other sizes, shapes, numbers and configurations of locking mechanisms are used in alternative embodiments. What is important is that a first feature(s) on top cap 52 engage with a corresponding second feature(s) on some portion of tube 54 so that swab assembly 5A may be desirably locked in place such as described herein.

Also as illustrated in FIG. 20, tube 54 secures vial 32′ via threads 54A inside of tube 54 and threads 54A′ on the top of vial 32′, which rotationally engage as will be understood to those of skill in the art. Preferably, vial 32′ (and threads 54A′), tube 54 (and threads 54A) have a notched feature as described in connection with the embodiments illustrated in FIGS. 9, 10, 14, 15, 16B, 16C, etc., such disclosure being directly applicable to the embodiment illustrated in FIG. 20. What is important is that gasket 30 engage with tube 54 and vial 32′ to provide a venting/sealing mechanism as described elsewhere herein.

Also, as illustrated in FIG. 20, indicator strip 27 is provided in the stem of swab assembly 5A, thereby providing an indication of adequate fluid absorption in swab 12A (sponge like material, as described elsewhere herein), or at least a confidence measure that sufficient fluid has been absorbed. Also as illustrated in FIG. 20, an analyte detecting strip 27′ (similar to strips 7 and other strips as described elsewhere herein) optionally is included in stem 6A of swab assembly 5A. Thus, two or more strips such as strip 27 and 27′ may be provided in stem 6A of swab assembly 5A. In one embodiment, only indicator strip 27 is present in stem 6A; in another embodiment, indicator strip 27 is present and in a back-to-back configuration strip 27′ also is present (e.g., a strip for detecting opioids or other drugs of abuse, or viruses or other analytes, antigens, antibodies, etc.). In yet another embodiment, two of strips 27′ are provided, preferably in a back-to-back configuration. In such embodiments, for example, one or more drugs or diseases may be detected in a rapid scan fashion by strips 27′, while vial 32′ captures an oral fluid sample for subsequent laboratory, confirmation or other testing. Thus, the embodiment of FIG. 20 encompasses alternatives including a rapid scan with strips, while capturing a sample for subsequent laboratory testing.

Also, the embodiment of FIG. 20 is contemplated for use in capturing a sample that may be used for genetic testing, genetic identification, virus or disease detection, drug or other analyte detection, etc., with or without strips 27/27′. All such disclosures relevant to such applications provided elsewhere herein should be understood to be as applicable to the embodiments described in connection with FIG. 20.

Also, it will be understood that vial 32 may be used a buffer or preservative material, such as preserving genetic material of cells, viruses, other pathogens or the like. In an exemplary workflow relevant to the embodiment of FIG. 20 (and also other embodiments), a user may be provided with tube 54 including gasket 30, an unused swab assembly 5A, and vial 32′ separate from tube 54 containing a liquid or solid buffer/preservative (e.g., for deactivating a virus while preserving RNA), preferably secured in vial 32′ with a screw cap (not shown, but well understood in the art). The user puts swab assembly 5A in his/her mouth to absorb oral fluid (preferably vigorously rubbing the tongue, back of the tongue, checks, inner lips, under the tongue, etc.) and cellular and other material, or which otherwise attach to swab 12A. Before, during or after the fluid, etc. absorption process, the user unscrews the cap, secures vial 32′ (containing the optional buffer/preservative) to tube 54 via threads 54A/54A′ in a rotational, know manner). After, for example, confirming adequate oral fluid absorption via indicator strip 27 (and/or via analyte strip 27′—which may include a rapid scan test of one or more target analytes, pathogens, etc.), then the user inserts swab assembly 5A into tube 54 and pushes down to engage tabs 58A and openings 58B (as previously described), with gasket 30 funneling fluid into vial 32′ in a first/venting position, which moving under pressure to a second/sealing position (all as described else where herein). The entire device may then be delivered to a laboratory (via hand delivery or a courier/delivery service), where the neat (unbuffered) or buffered/reserved sample in vial 32′ may be analyzed with laboratory instrument (e.g., LC MS) for confirmation, viral agent, genetic detection/analysis, etc. As will be appreciated, oral specimens may be readily obtained in a convenient manner in a variety of settings, with a rapid scan optionally provided, and with a sample ready for laboratory analysis such as for detecting viruses (e.g., influenza, COVID-19 or other coronaviruses, cancer, HIV, hepatitis, drugs of abuse, genetic information, or other markers or information obtainable via oral fluids).

Exemplary buffers for such a workflow include, but are not limited to, saline, saliva DNA preservation buffer, guanidinium thiocyanate (GTC) or guanidinium isothiocyanate (GITC) (which are chemical compounds which may be used as a general protein denaturant, being a chaotropic agent, although most commonly used as a nucleic acid protector in the extraction of DNA and RNA from cells), sodium citrate, a citrate-containing buffer, sodium azide, virus cell-lyse storage buffer (e.g., a buffer having a lysis effect on, for example, a virus, while preserving RNA information), and the like. Any such buffer/preservative, particularly for use in the workflow described above, are within the scope of embodiments of the present invention.

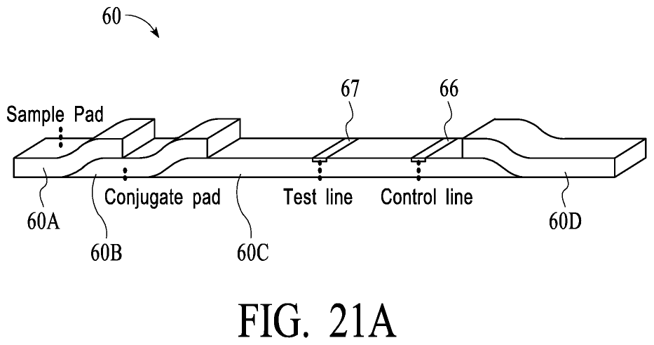

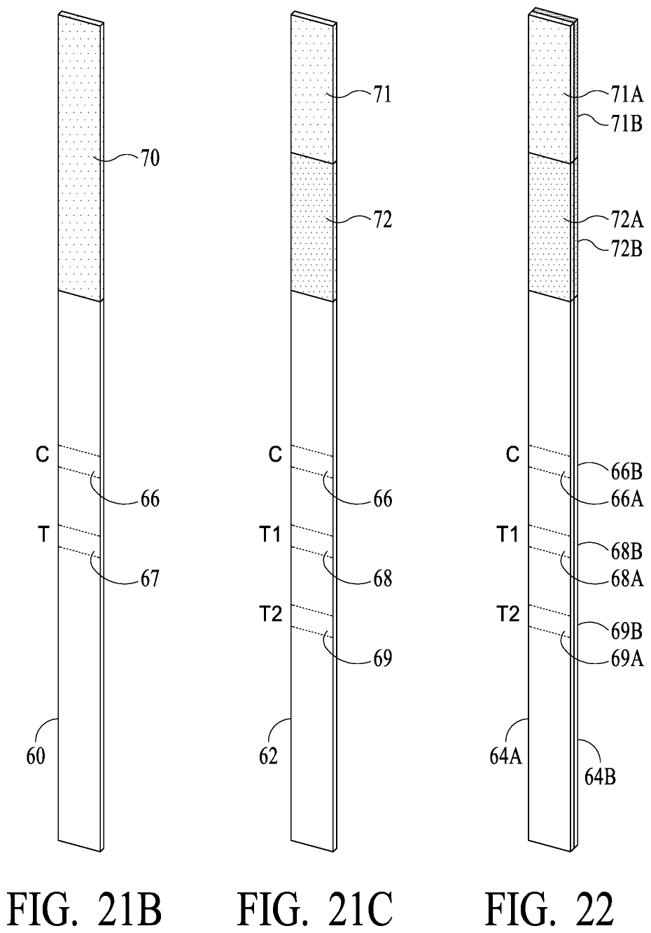

Referring now to FIG. 21A to FIG. 24C, and without being bound by theory, exemplary additional details of preferred embodiments of the present invention including a lateral flow, analyte detection strip in the stem of a swab assembly will now be described.

Lateral flow assays using analyte test strips are known in the art to provide disposable diagnostic devices that can test for drugs, diseases (e.g., viruses) and other biomarkers and analytes in samples such as saliva, blood, urine, and food. Embodiments of the present invention utilize a wide variety of such analyte test strips for the diagnosis of sexually transmitted diseases, mosquito-borne diseases, tuberculosis, hepatitis, pregnancy and fertility testing, cardiac markers, cholesterol/lipid testing, drugs of abuse, veterinary diagnostics, cancer markers, and food safety, all of which are within the scope of such embodiments.

Encompassed within the scope of embodiments of the present invention are lateral flow strips of assay formats called “sandwich” and “competitive.”. As will be understood by those of skill in the art based on the disclosures herein, a sandwich assay often is used for detecting larger analytes that have at least two binding sites. Typically, an antibody to one binding site is conjugated to the nanoparticle, and an antibody to another binding site is used for the test line of the assay. If there is analyte present in the sample, the analyte will bind to both the antibody-nanoparticle conjugate and to the antibody on the test line, yielding a positive signal. The sandwich format results in a signal intensity at the test line that is directly proportional to the amount of analyte present in the sample. Regardless of the quantity of analyte in the sample, an anti-species antibody at the control line will bind the nanoparticle, yielding a control line signal that demonstrates that the assay is functioning correctly.

The competitive format is used for detecting analytes when antibody pairs are unavailable or if the analyte is too small for multiple antibody binding events, such as steroids and drugs. In this format, the test line typically contains the analyte molecule, usually a protein-analyte complex, and the conjugate pad contains the detection antibody-nanoparticle conjugate. If the target analyte is present, the analyte will bind to the conjugate and prevent it from binding to the analyte at the test line. If the analyte is not present, the conjugates will bind to the analyte at the test line, yielding a signal. In the competitive format, the signal intensity is inversely proportional to the amount of analyte present in the sample. As in the sandwich format, the control line will bind the nanoparticle conjugate with or without the analyte providing confidence that the assay is working correctly.

A typical lateral flow test strip used in accordance with embodiments is illustrated in FIG. 21A. Strip 60 preferably includes sample pad 60A, conjugate pad 60B, strip body 60C, preferably a nitrocellulose or similar membrane-type strip that contains test and control lines 67 and 66, and also preferably includes wicking pad 60D. As illustrated, sample pad 60A preferably overlaps conjugate pad 60B, conjugate pad 60B preferably overlaps strip body 60C, and strip body 60C preferably overlaps wicking pad 60D. The overlap in general is determined in facilitate capillary flow of the specimen from sample pad 60A through the strip toward wicking pad 60D, as will be appreciated by those of skill in the art.

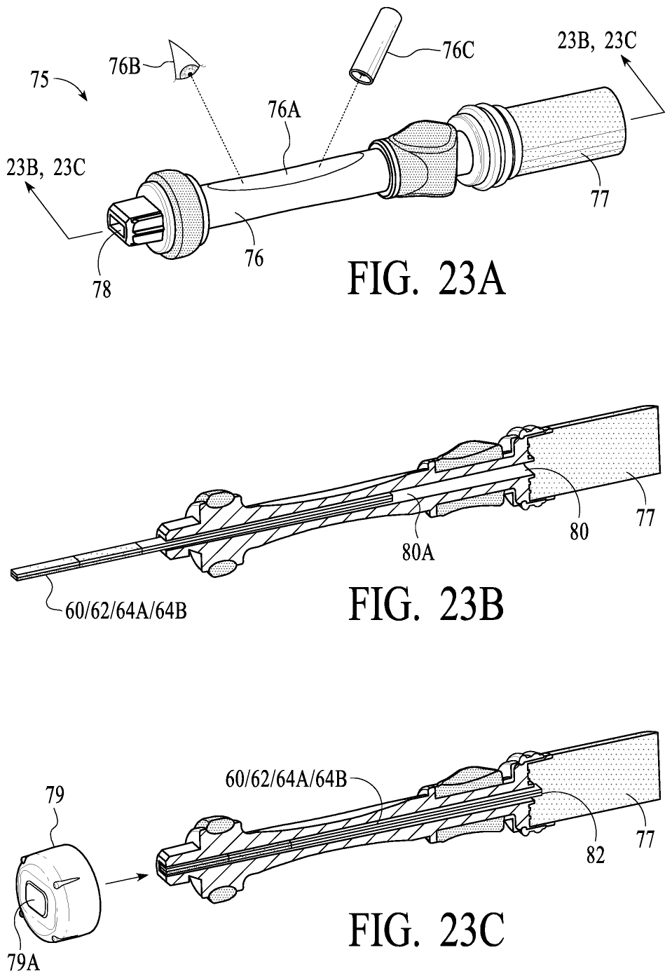

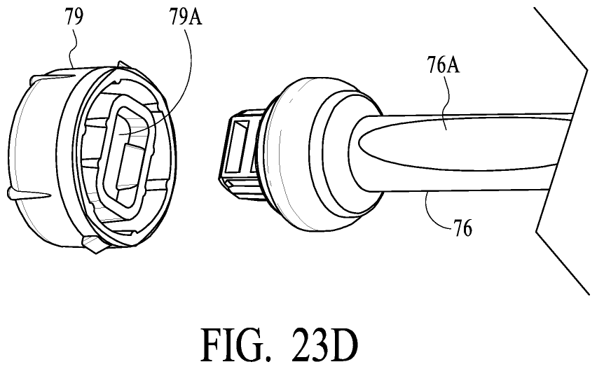

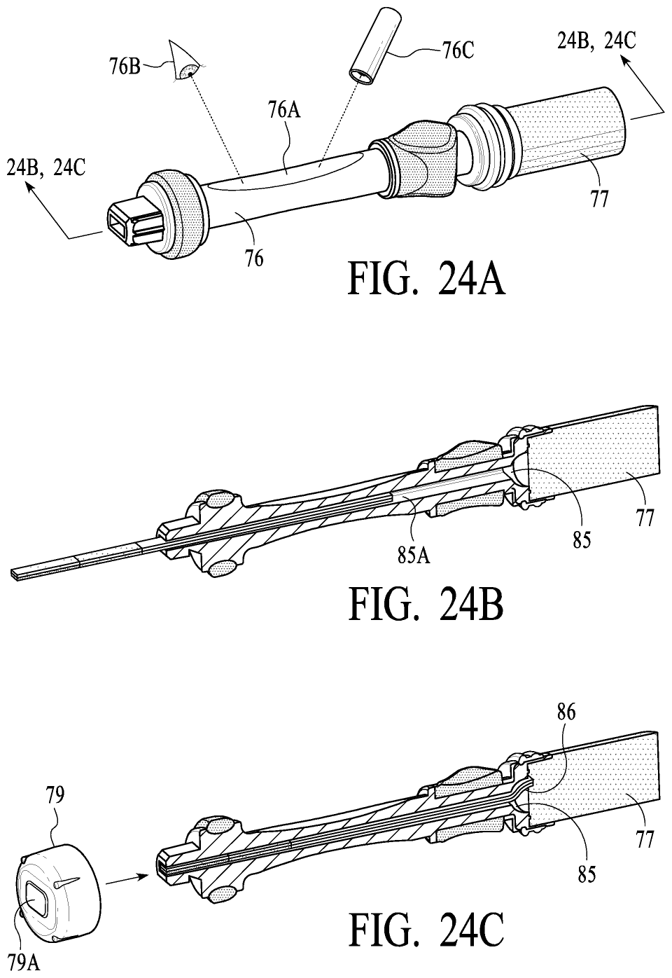

In accordance with embodiments of the present invention, preferably a saliva specimen is presented (or flows) to sample pad 60A, either by compression of an oral swab in accordance with the embodiments of FIGS. 1-19, or by way of saliva penetrating swab or sponge/absorbent material to reach sample pad 60A in accordance with certain embodiments of FIG. 20 or the embodiments of FIGS. 23A-24C, and as will be understood by those of skill in the art, fluid from the specimen wicks through strip 60. Sample pad 60A preferably neutralizes the specimen and preferably filters unwanted particulates that may reach sample pad 60A. This enables fluid from the specimen to flow to conjugate pad 60B, which preferably contains colored or fluorescent nanoparticles that preferably have an antibody on their surface. When the fluid of the specimen reaches conjugate pad 60B, dried nanoparticles are released and mix with the fluid of the specimen. Any target analytes in the fluid of the specimen that correspond to the antibody, these preferably bind to the antibody and flow with the specimen through strip body 60C and across one or more test lines 67 and control line 66 as illustrated. Test line 67 illustrated in FIG. 21A preferably is the primary read-out of the lateral flow assay and consists of immobilized proteins that can bind to the nanoparticle to generate a visually or optically perceptible signal that is correlated to the presence of the target analyte in the fluid of the specimen. The fluid of the specimen continues to flow along strip body 60C and eventually reaches control line 66. Control line 66 preferably contains affinity ligands (a ligand being a molecule or atom which binds reversibly to a protein, and can be an individual atom or ion, and can also be a larger and more complex molecule made from many atoms) that will bind with the nanoparticle conjugate whether or not there is analyte present in the fluid of the specimen, and as will be understood by those of skill in the art serves to confirm that the lateral flow assay has worked correctly (an absence of a signal at control line 66 providing an indication that the test result is, for example, invalid). After control line 66, the fluid of the specimen continues to flow through strip body 60C until it reaches wicking pad 60D. In preferred embodiments, wicking pad 60D is provided to absorb sample liquid to ensure that there is consistent flow across test line(s) 67 and control line 66. After fluid of the specimen pass across test and control lines 67 and 66, lines will selectively be visible at test line(s) 67 and control line 60, and a user (directly or with the aid of an instrument such as a camera, smartphone with a camera, optical sensor, etc.—see user visual element 76B and/or camera/optical sensor 76C of FIGS. 23A and 24A) may read or determine the results. Also, as will be more fully appreciated by those of skill in the art, two (or more) analytes may be detected by appropriate selection of nanoparticles and antibodies so that two (or more) test lines 67 may be provided (see, also, the discussion in relation to embodiments referencing a plurality of such test lines). It will be understood that all embodiments using such a lateral flow assay strip may test one, two or more than two target analytes in accordance with the present invention.

FIG. 21B illustrates in a more schematic form lateral flow strip 60 (which may have constituent elements as described in connection with FIG. 21A) having a single test line 67 and control line 66, and also analyte label area 70. Label area 70 preferably provides a visual indication of the target analyte for the strip, which could be a drug (e.g., METH for methamphetamines. COC for cocaine, Ag for antigens, AB for antibodies, etc.), and which may by way of letters and background colors or patterns that facilitate user or camera/optical instrument reading of test results from the strip. FIG. 21C illustrates in a more schematic form lateral flow strip 62 (which may have constituent elements as described in connection with FIG. 21A, but of the form for two analytes) having two test lines 67 and control line 66, and also analyte label areas 71 and 72, which preferably provides a visual indication of the target analytes for the strip, which may by way of letters and background colors or patterns that facilitate user or instrument reading of test results from the strip.

In accordance with certain embodiments illustrated in FIGS. 20 and 23A-24C, strips may be provided in stem 6A (FIG. 20) or 76A (FIGS. 23A-24C) and be presented with fluid of the specimen via contact with swab 12A (FIG. 20) or 77 (FIGS. 23A-24C). Strips provided in the embodiments of FIGS. 23A-24C may include one strip with one target analyte (see, e.g., FIG. 21B), or it may include one strip with two or more target analytes (see, e.g., FIG. 21C), or it may include two strips with one or two or more target analytes. The embodiment of FIG. 22 illustrates a back-to-back arrangement of two strips 64A and 64B, each with two target analytes test lines 68A, 68B, 69A and 69B and a control line 66A, 66B, and analyte label areas 71A, 71B, 72A, 72B. It should be understood that one, two or three target analyte strips are within the scope of the present invention, and such back-to-back strips as illustrated in FIG. 22 are illustrative. A single analyte test strip could be paired with a two or three analyte test strip, and a two analyte test strip could be paired with a three analyte test strip. In one preferred embodiment, a viral antigen (Ag) single analyte test strip is provided on a first side of a back-to-back strip configuration, and one or two antibody (AB) analyte test strip is provided on a second side of a back-to-back strip configuration (e.g., IgG and IgM antibodies to SARS-CoV-2). Also, in accordance with alternative embodiments a stem may contain an indicator strip (such as indicator strip 27 described elsewhere herein) in a back-to-back configuration with a test strip of one, two or more target analytes. Also, channels within the swab stem (see e.g., channel 80A of FIG. 23B and channel 85A of FIG. 24B) preferably is a rectangular volume to accommodate back-to-back strip configurations, other geometries are possible to accommodate more strips in the swab stem (e.g., three or more strips in a circular/tubular arrangement in the swab stem). What is important in such alternative embodiments is that one or more lateral flow test strips for one, two or more target analytes are positioned in stem 5A/76 and be in fluid contact with the specimen via swab 12A/77 such that the lateral flow assay runs with results visible to a user or instrument via a window in stem 5A/76 (e.g., window 6A, 76A). An oral swab-type implement may be then be obtained with one or a plurality of lateral flow test strips in the stem of the swab, such that the swab may be inserted into the mouth, absorb fluid, and analytes detected via the test and control lines and be read much like an old-school, conventional thermometer.

In addition, as will be appreciated with the embodiments of FIG. 20, one or more target analyte test strips may be included in the swab stem, which may be produced by having a swab stem sealed at the upper portion receiving a stem cap, with the one or more test strips inserted into a channel in the stem and fixed in place with the swab sponge being adhered to the swab stem such as via an adhesive. As illustrated in FIGS. 23A-24C, in alternative embodiments the swab sponge-type material is adhered to the swab stem and a later time one or more analyte to test strips are inserted into a channel of the stem (see, e.g., channel 80A of FIG. 23B and channel 85A of FIG. 24B) and inserted so as to contact the swab material (e.g., swab 77 of FIGS. 23C and 24C) and be brought into fluid contact with the specimen. In such embodiments, and as illustrated in greater detail in FIG. 23D, cap 79 is configured to have seal 79A (which preferably is a pliable material such as rubber or silicon), and as cap 79 is secured onto swab 75 by one more holding features (e.g., an indentation on swab stem 76 and a protrusion on cap 79 configured such that the indentation snaps into the protrusion so that seal 79A provides a preferably airtight seal over opening 78 (which is the opening into which test strips are inserted—see, e.g., FIGS. 23B and 24B). In one alternative embodiment, the protrusion/indentation features illustrated in FIG. 23D are substituted with corresponding threads on cap 79 and step 76 such that the cap is screwed down bringing seal 79A into position to seal or close opening 78. What is important is that cap 79 has a sealing feature that covers/seals opening 78 after test strips 60/62/64A/64B, etc. are fully inserted into channel 80A or 85A of swab stem 76.

The exemplary embodiment of FIG. 24C differs from the embodiment of FIG. 23C in that channel 85A has end portion/cavity 85 with a larger open volume as compared to the body of channel 85A/80A, which preferably have a curved inner surface. As illustrated, in such preferred embodiments test strips 60/62/64A/64B have a first length that is longer than the length of the channel in swab step 76 (second length). With a first length (strip length) longer that the second length (channel length from beginning to end), greater contact between the test strips 60/62/64A/64B and swab absorbent material 12A/77 may be achieved, which preferably improves flow/communication of the fluid of the specimen to the sample pad of the test strip (FIG. 21A). With the open and preferably curved inner surface of cavity 85 as illustrated, a volume is provided such that the test strip may bend or fold in this volume, and thereby inhibit bending or folding at another point in the channel, and thereby helping to present the test strip to fluid of the specimen.

In one alternative embodiment, the holding feature of the cap onto the swab stem may be by way of threads, such as (for example) a screw on cap with threads that correspond to threads on the tip of stem 76, with seal 79A being a seal inside the cap that seals opening 78 % ben the cap is screwed onto the swab stem. In yet another embodiment, cap 79 has an opening without seal 79A, and the opening of cap 79 is configured so that it may be covered by (for example) a thumb of a user so that if the swab is pushed into a compression housing or tube (such as described elsewhere herein) the hole is sealed by the users thumb, thereby improving compression and remove of fluid into a specimen vial as described elsewhere herein.

In yet another alternative embodiment, strips with one or more target analytes are combined with the embodiments of FIGS. 1-19, so that target analytes may be tested via strips in a cassette of such embodiments, and also in the swab stem. In one such alternative, the strip in the swab stem tests for THC (from marijuana), either the parent molecule (Δ(9)-tetrahydrocannabinol (THC), the primary psychoactive component in cannabis or marijuana) or a THC metabolite such as 11-hydroxy-THC (11-OH-THC) or 11-nor-9-carboxy-THC (THCCOOH), and in one embodiment includes two strips in a back-to-back configuration with one target analyte on a first strip being the THC parent molecule, and a second target analyte on a second strip being a THC metabolite. In another alternative embodiment, an alcohol test strip as is known in the art is included in the swab stem and a plurality of drugs of abuse are detected via a plurality of test strips in the cassettes of one of the embodiments of FIGS. 1-19.

In yet another embodiment, lateral flow test strips such as are described in the embodiments illustrated in FIGS. 1-19, FIG. 20, or FIGS. 21A-24C, a target analyte may be a viral antigen (Ag), such a virus being, for example, a coronavirus. Ag detection has been observed with neat saliva specimens (saliva only with no buffer. VTM or other medium or agents), but in alternative preferred embodiments a sample pad (see FIG. 21A) is pre-treated with a releasing-type or surfactant-type material or a detergent type material, such as Triton X-100, or a non-ionic detergent such as TWEEN-20, which has been determined to improve antigen detection in certain circumstances. Such pre-treatment of a test strip sample pad may be made by pressing a solid material into the material of the sample pad, or by a slurry/solution type process preferably with a fast evaporating solvent such as a type of alcohol (where a small amount of slurry/solution containing the releasing agent is applied to sample pad (preferably at a lower portion away from the overlap area with the conjugate pad), under conditions in which the carrying liquid/solvent evaporates quickly and/or is of insufficient volume to flow up the test strip to reach the conjugate pad, or alternatively is applied to the sample pad and liquid/solvent is removed before the sample pad is overlapped with the conjugate pad. What is important is that a controlled amount of the releasing-type agent is applied to/included with the sample pad of the test strip so as to not interfere with the working of the conjugate pad, such that antigen detection is enhanced when the fluid specimen contacts the sample pad and begins capillary flow through the test strip.

As will be appreciated from the disclosures herein, exemplary preferred embodiments of the present invention include a fluid sample testing apparatus having a fluid collector with an elongated stem and an absorbent member affixed to a first end of the stem, the stem having an interior open channel extending from the first end of the stem to a distal second end of the stem. One or more lateral flow assay strips may be positioned in the channel of the stem, and a sample reception member of each of the one or more lateral flow assay strips is in fluid communication with the absorbent member. One or more test lines and a control line of the one or more lateral flow strips are visible through a window portion of the stem. Fluid introduced into the absorbent member reaches the sample reception member of the one or more lateral flow assay strips and a test result is perceptible by a user or instrument via the one or more test lines and the control line via the window portion of the stem. At least two lateral flow assay strips are positioned in the channel of the stem, and each of the at least two lateral flow assay strips may be adapted to detect the presence or absence of at least two analytes. Each of the one or more lateral flow assay strips may be adapted to detect the presence or absence of one or more analytes, which may be one or more drugs, an antigen or antibody corresponding to a virus, or other substances, biomarkers or the like as disclosed elsewhere herein. As one example, a first lateral flow assay strip may test for an antigen corresponding to the virus, and a second lateral flow assay strip may test for an antibody corresponding to the virus. The swab assembly may be used with a fluid collector (or compression) tube, and a sample holding container (or vial) may be provided in fluid communication with the fluid collector tube. The fluid collector preferably is adapted for insertion into the fluid collector tube and upon insertion and penetration into the fluid collector tube pressure is generated to release fluid from the fluid collector into the sample holding container. Fluid contained in the sample holding container preferably has sufficient volume for a confirmation test or a disease testing panel in a laboratory. In one example, lateral flow strips provide rapid results of antigen and antibody testing, and the specimen in the vial is transported to a laboratory for rtPCR or other molecular testing for a virus (such as coronavirus) and also for additional testing, such as respiratory test panel for pneumonia, influenza and other diseases.

The channel of the stem may be closed or open at the second end of the stem. The channel of the stem may be closed at the second end of the stem via a cap affixed to the second end of the stem, which may be via one or more projections/indentations on the cap and stem or via threads correspondingly formed on the cap and stem. A seal preferably is provided to seal the hole, or the cap and stem may be configured so that a body part of the user (e.g., a thumb) seals the hole to facilitate compression of the swab assembly in a compression tube. A fluid indicator strip may be positioned in the channel of the stem along with one or more lateral flow test strips. Multiple swab assemblies or fluid collectors may be provided along with a housing with a fluid collector tube and one or more second lateral flow assay strips in fluid communication with the fluid collector tube. Each fluid collector may be adapted for insertion into a fluid collector tube and upon insertion and penetration into the fluid collector tube pressure is generated to release fluid from the fluid collector, and the fluid may be presented to the one or more second lateral flow strips which may be in cassette, and the presence or absence of one or more analytes in the sample may be detected based on the lateral flow assay strips in the channel of the stem and based on the second lateral flow assay strips. The swab assemblies/lateral flow strip configurations of FIGS. 20-24C may be used with the housing/vial-specimen containers/lateral flow strip cassettes of FIGS. 1-19.

Preferably the channel in the swab stem has a first length, and the one or more lateral flow assay strips are of at least a second length, and preferably the second length is greater than the first length. Preferably, one or more of the lateral flow assay strips detect the presence or absence of a virus antigen, and one or more of the lateral flow assay strips have a sample pad in fluid communication with the absorbent member, and the sample pad includes an agent that facilitates antigen detection via the one or more lateral flow assay strips. The agent may including a detergent, which may include Triton, Triton X-100. TWEEN 20, non ionic or other detergents, as well as other substances that facilitate antigen release/detection.

The present invention contemplates methods with steps such as absorbing a fluid sample to be tested on an absorbent member affixed to a first end of an elongated stem of a fluid collector, which may be by a user putting the fluid collector or swab in the mouth or otherwise in contact with the fluid to be tested. In one alternative embodiment, a swab sponge/absorbent type material may be wetted with water or saline solution and an unknown substance (e.g., a powder or residue) may be scrubbed or wiped up with the sponge/absorbent type material so that lateral flow strips may detect if the substance is a drug or other material containing a target analyte. The stem preferably has an interior open channel extending from the first end of the stem to a distal second end of the stem, and one or more lateral flow assay strips are positioned in the channel of the stem. A sample reception member of each of the one or more lateral flow assay strips preferably is in fluid communication with the absorbent member, wherein one or more test lines and a control line of the one or more lateral flow strips are visible through a window portion of the stem. Fluid introduced into the absorbent member may then reach the sample reception member of the one or more lateral flow assay strips and a test result may be determined via visual user perception or an instrument such as a camera, smartphone camera or optical sensor via the one or more test lines and the control line via the window portion of the stem.

As will be understood, various testing methods are available based on the embodiments of FIGS. 20-24A, alone or in combination with the embodiments of FIGS. 1-19.

As will be understood by those of skill in the art, teachings from co-pending US App. Ser. Nos. 15/417,905, which has been incorporated by reference herein, are generally applicable to the exemplary preferred embodiments set forth herein. By way of additional explanation and not by way of limitation, certain of such teachings are set forth below.

### Analyte Screening

An embodiment of the present invention provides an analyte screening device which includes a rapid screening, lateral flow chromatographic immunoassay for the simultaneous, qualitative or quantitative detection of analytes in a fluid sample. For example, without limitation, the fluid sample may be saliva, urine, blood, mucus, water, or fluid extract of a solid or a semi-solid, for example stool or mucus or liquid biopsy. The fluid sample may also be an environmental sample, for example, without limitation, soil, dust, water, plant matter, insect, animal matter, or a fluid extract of any of the foregoing. The fluid sample may also be a food or beverage, for example, without limitation, a liquid beverage, a liquid-containing food, or a fluid extract of a solid, semi-solid or powdered food or beverage. The fluid sample may also contain genomic or proteomic material for testing and analysis.

An embodiment of the invention includes at least one membrane test strip, in fluid communication with a sample receiving member, able to indicate the presence or absence of at least one analyte above or below a threshold concentration in the fluid sample using a lateral flow chromatographic assay.

In an embodiment of the invention, the lateral flow chromatographic assay is a competitive assay, in which an analyte in the fluid sample competes with a competitor for binding with an anti-analyte antibody. For example, the anti-analyte antibody may be labeled, and the competitor may be immobilized in the test region of the membrane test strip. After the fluid sample reaches the dye region, it encounters the labeled anti-analyte antibody. If the analyte is present in the fluid sample above a predetermined threshold concentration, the analyte will saturate the binding sites of the labeled anti-analyte antibody; otherwise, some or the entire labeled anti-analyte antibody remains free to bind the competitor. As the fluid sample migrates along the membrane test strip by capillary action, it carries the labeled anti-analyte antibody along until it reaches the test region. The test region contains the immobilized competitor, which may be the analyte, fragments of the analyte, epitopes of the analyte, molecular mimics of the analyte, anti-idiotypic antibodies, or any other molecule able to compete with the analyte for binding to the anti-analyte antibody. If the analyte is present above the predetermined threshold concentration, the labeled anti-analyte antibody is saturated and does not bind the immobilized competitor, resulting in no signal in the test region; otherwise, the anti-analyte antibody is unsaturated and can bind to the competitor, resulting in a signal in the test region.

Thus, according to an embodiment of the invention employing a competitive assay, an analyte-negative fluid sample (containing lower than the predetermined concentration of the analyte) will generate a line in the test region due to capture of the labeled anti-analyte antibody, whereas an analyte-positive fluid specimen will not generate a colored line in the test region because the analyte in the fluid sample will saturate the labeled antibody and thus prevent its capture in the test region.

In an embodiment of the invention, the lateral flow chromatographic assay is a sandwich assay, in which the analyte must be present for the labeled anti-analyte antibodies to be captured in the test region. For example, the analyte antibody may be a labeled antibody, and a second anti-analyte antibody may be immobilized in the test region. For example, after the fluid sample reaches the dye region, it encounters the labeled anti-analyte antibody. If the analyte is present in the fluid sample, it will bind at least a fraction of the labeled anti-analyte antibody. As the fluid sample migrates along the membrane test strip by capillary action, it carries the labeled anti-analyte antibody along until it reaches the test region. The test region contains an immobilized anti-analyte antibody, which may be reactive against a different epitope of the analyte than the labeled anti-analyte antibody. If the analyte is present in the fluid sample, it forms a scaffold through which the labeled antibodies are immobilized in the test region. The fraction of the labeled antibodies captured in the test region is thus determined by the concentration of analyte in the fluid sample. If the analyte of interest is present above a predetermined threshold concentration, a sufficient fraction of the labeled antibodies are captured, resulting in a visible signal in the test region; otherwise, an insufficient fraction of the antibodies are captured and no signal is visible in the test region.

Thus, according to an embodiment of the invention employing a sandwich assay, an analyte-positive fluid specimen will generate a colored line in the test region of the membrane test strip due to the capture of the labeled antibody in the test region, whereas an analyte-negative fluid sample will not generate a fine in the test region due to failure to capture the labeled antibody.

Embodiments of the invention include a positive control to indicate that the assay has functioned properly and is complete. For example, the dye region may include a labeled control protein, including without limitation a labeled control antibody, and the control region of the membrane test strip may contain an immobilized control agent able to capture the labeled control protein, such as an antibody or a control analyte. The control region may be located distal to each test region on the membrane test strip, such that the fluid sample will encounter each test region before encountering the control region. The reaction of the labeled control protein with the immobilized control agent produces a colored line in the control region, indicating that a proper volume of the fluid sample has been added and membrane wicking has occurred, and the assay has worked properly.

An embodiment of the invention concurrently tests for multiple analytes, for example by employing membrane test strips capable of testing multiple analytes concurrently (for example, by containing multiple anti-analyte antibodies in the dye region and having multiple compatible test region), and/or by employing multiple membrane test strips within the same apparatus. An embodiment of the invention includes both membrane test strips that employ a competitive assay and a sandwich assay, for example on different membrane test strips within the device and/or on the same membrane test strip within the device.

Embodiments of the invention may provide quantitative determination of the concentration of an analyte that is present in the fluid sample. For example, the apparatus may include multiple membrane test strips having varying amounts of an anti-analyte antibody, resulting in varying analyte sensitivity, such that the concentration of the analyte is indicated by which of the membrane test strips show or fail to show a colored line in the test region.

### Antibodies

An embodiment of the invention employs antibodies for the detection of analytes. The term “antibody” (Ab) as used herein includes monoclonal antibodies, polyclonal antibodies, multispecific antibodies (for example bispecific antibodies), and antibody fragments, so long as they exhibit the desired activity. The term “monoclonal antibody” as used herein refers to an antibody obtained from a population of substantially homogeneous antibodies, that is, the individual antibodies comprising the population are identical except for possible naturally occurring mutations that may be present in minor amounts.

The terms “labeled antibody” and “labeled control protein” refer to an antibody or protein that is conjugated directly or indirectly to a label. The label is a detectable compound or composition that may be detectable by itself, Including without limitation a dye, colloidal metal (including without limitation colloidal gold), radioisotope, or fluorescent compound, or, in the case of an enzymatic label, may catalyze chemical alteration of a substrate compound or composition which is detectable, or any combination of the foregoing.

### Analytes

According to an embodiment of the invention, the apparatus includes a device for testing a fluid sample for the presence of analytes. The present invention contemplates testing for any analyte. Without limitation, analytes that may be tested for include drugs of abuse or their metabolites, analytes indicating the presence of an infectious agent or product of an infectious agent, allergen, pollutant, toxin, contaminant, analyte with diagnostic or medical value, antibody against any of the foregoing, and any combination thereof.

According to an embodiment of the invention, analytes that may be tested for include drugs of abuse and their metabolites, including without limitation 7-acetaminoclonazepam, alkyl nitrites, alpha-hydroxyalprazolam, alprazolam, 2-amino-2′-chloro-5-nitrobenzophenone, 7-aminoclonazepam, 7-aminonitrazepam, amitriptyline, amobarbital, amoxapine, amphetamine, anabolid steroids, androgen, androstadienone, aprobarbital, atropine, barbiturates, benzodiazepines, benzoylecgonine, benzylpiperazine, boldenone undecylenate, 4-bromo-2,5-dimethoxyphenethylamine, bovine growth hormone, butabarbital, butalbital, butripryline, 4-chlordehydromethyltestosterone, chloroform, clomipramine, clonazepam, clostebol, cocaethylene, cocaine, codeine, codeine-6-glucuronide, cotinine, dehydroepiandrosterone, desipramine, desmethyldiazepam, desoxymethyltestosterone, dexmethylphenidate, dextroamphetamine, dextromethorphan, dextropropoxyphene, dextrorphan, 2,5-diamino-2′-chlorobenzophenone, diamorphine, diazepam, dibenzepin, dihvdrotestosterone, dimenhydrinate, 2,5-dimethoxy-4-(n)-propylthiophenethylamine, 2,5-dimethoxy-4-ethylphenethylamine, 2,5-dimethoxy-4-iodophenethylamine, dimethyl ether, dimethyltryptamine, dimethyltryptamine, diphenhydramine hydrochloride, dosulepin hydrochloride, dothiepin hydrochloride, doxepin, drostanolone, ecgonine, ecgonine methyl ester, ephedrine, ergine, estren, 5-estrogen, ethyl-5-(1′-methyl-3′-carboxypropyl)-2-thiobarbituric acid, 5-ethyl-5-(1′-methyl-3′-hydroxybutyl)-2-thiobarbituric acid, ethylestrenol, ethylphenidate, fentanyl, flunitrazepam, fluoxymesterone, furazabol, gamma-hydroxybutyrate, 1-(beta-D-glucopyranosyl) amobarbital, growth hormone, heroine, hexabarbital, human chorionic gonadotropin, human growth hormone, hydrocodone, hydromorphone, (+)-3-hydroxy-N-methylmorphinan, 3-hydroxy clonazepam, 11-hydroxy-tetrahydrocannabinol (I 1-hydroxy-THC), 3′-hydroxyamobarbital, p-hydroxyamphetamine, p-hydroxynorophedrine, imipramine, iprindole, kava, katamine, levomethylphenidate, iofepramine, lorazepam, lorazepam-glucuronide, lysergic acid diethylamide, meperidine, mescaline, mestanolone, mesterolone, meta-chlorophenylpiperazine, methadone, methamphetamine, methandrostenolone, methcathinone, 3,4-methylenedioxyamphetamine, methanolone, methanolone enanthate, methylenedioxymethamphelamine (ecstacy), methylphenidate, methylphenobarbital, methyl testosterone, mibolerone, (+)-3-morphinan, morphine, nandrolone, nicotine, nitrazepam. N-methyl-diethanciamine, norbolethone, norcodeine, norethandrolone, norketamine, nortriptvline, opiates, opipramol, opium, oxabolone opionate, oxandrolone, oxazepam, oxvcodone, oxymetholone, oxymorphone, pentobarbital, phencyclidine, phenethylamines, phenobarbital, 4-phenyl-4-(1-piperidinyl)-cyclohexanol, 1-phenyl-1-cyclohexene, phenylacetone, 5-[N-(1-phenylcyclohexyl)]-aminopentanoic acid, 1-(1-phenylcyclohexyl)-4-hydroxypiperidine, piperidine, protriptyline, psilocin, psilocvbin, quinbolone, salvinorin A, scopolamine, secobarbital, sodium thiopental, stanozolol, telbutal, temazepam, testosterone, testosterone proprionate, tetrahydrocannabinol (THC). THC-COOH, tetrahydrogestrinone, toluene, trenbolone, tricyclic antidepressant, 3-trifluoromethylphenylpiperazine, trimipramine, tryptamines, or any combination thereof. The minimum concentration level at which the presence of any particular drug or metabolite is detached may be determined by various industry minimum standards, such as, for example, the National Institute on Drug Abuse (NIDA), the Substance Abuse & Mental Health Services Administration (SAMHSA), and the World Health Organization (WHO).

According to an embodiment of the invention, analytes that may be tested for include infectious agent or the products of an infectious agent, including without limitation Acanthamoeba, aflatoxin, alimentary mycotoxlcoses, altertoxin, amoeba, Anisakis, Ascaris lumbricoides, Bacillus arthracis, Bacillus cereus or its toxin, bacteria, bovine spongiform encephalopathy prioris, Brucella, Caliciviridae, Calymmatobacterium granulomatis, Campylobacter, Campylobacter jejuni, Candida. Candida albicans, Cephalosporium, Chlamydia trachomatis, chronic wasting disease prions, Citrinin, Clostridium botulinum or its toxin, Clostridium perfringens, Corynebacterium ulcerans. Coxielia burnetil, Creutzieldt-Jakob disease prions, Cryptococcus neoformans. Cryptosporidium, Cryptosporidium parvum Cycloplazonic acid, Cyclospera cayetanensis, Cytochaiasin, Cytomegalovirus, Diphyilobothrium, Escherichia Coli. Ebola, endotoxin, Entarnceba histolytica, Enterovirus, Ergopeptine alkaloid, Ergot alkaloid, Ergotamine, Escherichia coli O157. Eustrongylides, Fasciola hepatica, fatal familial insomnia prions, flatworm, Francisella tularensis. Fumitremorgen B.sub.I Fumonisin, Fusarium, Fusarochromanone, genital warts, Gerstmann-Straussler-Scheinker syndrome prions, Giardia, Giardia lamblia, Granuloma inguinale, H7 enterohemorrhagic, Haemophilus ducreyl, Helicobacter pylori, Hepatitis, Hepatitis A, Hepatitis B, Hepatitis C. Hepatitis D, Hepatitis E, Hepatitis E, herpes simplex virus, Histoplasma capsulatum, HIV, HIV-1, HIV-2, human papillomavirus, influenza, Kaposi's sarcoma-associated herpesvirus, Kojic acid, kuru prions, Listeria monocytogenes. Lolitrem alkaloids, marburg virus. Methicillin-resistant Staphylococcus aureus or its toxin, molluscum, Moniliformin, mononucleosis, mycobacteria, Mycobacterium tuberculosis, Mycoplasma, Mycoplasma hominis, Mycotoxins, Myrothecium, Nanophyetus, Neisseria gonorrhosae, nematode, Nivalenol, Norovirus. Oohratoxins, Oosporeine, parasite, Patulin, Paxilline, Penitrem A, Phomopsins, Plasmodium, Platyhelminthes, Plesiomonas shigelloides, Pneumococcus, Pneumocystis jirovecii, prions, protozoa, rhinovirus, Rotavirus. Salmonella, Sarcocystis hominis, Sarcocystis sulhominis, scraple prions, sexually transmitted disease, Shigella, Shigella, Sporidesmin A, Stachybotrys, Staphylococcus aureus or its toxin, Sterigmatocystin, Streptococcus, Streptococcus pneumoniae, Streptococcus pyogenes, Taenia saginata, Taenia solium, tapeworm. Tenia solium, Tinea, Toxoplasma gondii, Tremorgenic mycotoxins, Treponema palidum, Trichinella spiralis, Trichoderma, Trichomonas vaginalis, Trichothecene, Trichuris trichlura, Typanosoma cruzi, Ureaplasma urealyticum, Verrucosidin. Varruculogen, Vibrio cholerae non-O1, Vibrio cholerae O1, Vibrio-parahaemolyticus, Vibrio vulnificus, viruses, yeast infections, Yersinia enterocolitica, Yersinia pseudotuberculosis, Zearalenois, Zearalenone, antibodies against any of the foregoing, or any combination thereof. According to an embodiment of the invention, the analytes to be tested for include allergens, including without limitation aesculus, aider, almonds, animal products, artemisia vulgaris, beans, bet sting venom, birch, calyx, cat dander, celeriac, celery, Chenopodium album, cockroach, corn, dander, dong dander, drugs, dust mite excretion, egg albumen, eggs, Fei d 1 protein, fruit, fur, grass, hazel, hornbeam, insect stings, latex, legumes, local anaesthetics, maize, metal, milk, mold spores, mosquito saliva, mouse dander, nettle, olea, peanuts, peas, pecans, penicillin. Plant pollens, plantago, platanus, poplar, pumpkin, ragweed, rat dander, ryegrass, salicylates, seafood, sesame, sorrel, soy, soybeans, sulfonamides, tilia, timothy-grass, tree nuts, trees, wasp sting venom, weeds, wheat, willow, antibodies against any of the foregoing, or any combination thereof.

According to an embodiment of the invention, the analytes to be tested for include pollutants, toxins, and contaminants, including without limitation 1,2-Dibromoethane, acrylamide, aldehydes, arsenic, artificial growth hormone, asbestos, benzene, benzopyrene, carcinogens, dichloro-diphenyl-trichloroethane, formaldehyde, kepone, lead, mercury, methylmercury, nitrosamines, N-nitroso-N-methylurea, organochlorine insecticides, pesticides, polychlorinated biphenyls, polychlorinated dibenzofurans, polychlorinated dibenzo-p-dloxins, recombinant bovine growth hormone, recombinant bovine somatotropin, toluene, vinyl chloride, antibodies against any of the foregoing, or any combination thereof.

According to an embodiment of the invention, the analytes to be tested for include analytes with diagnostic or medical value, including without limitation acid phosphatase, active-B12, AFP, Alanine Aminotransferase, Alanine Aminotransferase, Albumin. Albumin BCG, Albumin BCP, Alkaline Phosphatase, Alpha-1 Antitrypsin, Alpha-1 Glycoprotein, Amikacin, Ammonia, Amylase, Anti-CCP. Anti-Tg, Anti-TPO, Apolipoprotein A1, Apolipoprotein B, ASO, Asparate Aminotransferase, Aspartate Aminotransferase, B12. Beta2 Microglobulin, Beta2 Microglobulin, BNP, CA 125, CA 125 II, CA 15-3, CA 19-9 XR, Calcium, Carbamazepine, Carbon Dioxide, CEA, Ceruloplasmin, Cholesterol, CK-MB, Complement C3, Complement C4, Cortisol, C-Peptide, C-Reactive Protein, Creatine Kinase, Creatinine, CRP Vario, Cyclosporine, Cyclosporine and Metabolite-Whole Blood, Cyclosporine Monoclonal-Whole Blood, D-Dimer, DHEA-S, Digitoxin, Digoxin, Digoxin, Digoxin II, Digoxin III, Direct Billirubin, Direct LDL, Estradiol, Ferritin, FLM II, Folate, Free Carbamazepine, Free Phenvtoin, Free PSA, Free T3, Free T4, Free Valproic acid, FSH, Gamma-Glutamyl Transferase, Gentamicin, Glucose, Glycated Hemoglobin, Haptoglobin, hCG, Hemoglobin, Homocysteine, ICT CI-, IGFBP-1, Immunoglobulin, Immunoglobulin A, Immunoglobulin E, immunoglobulin G, Immunoglobulin M, Insulin, Intact PTH, Iron, K+, Kappa Light Chain, Lactate Dehyrogenase. Lactic acid, Lambda Light Chain, LH, Lidocaine. Lipase, Lithium, Lp, magnesium, metabolites, Methotrexate II, Microalbumin, MPO, Myoglobin, Na-f, N-Acetyl-procainamide, neonatal Billirubin, NGAL, P-Amylase, Pepsinogen I, Pepsinogen 11, Phenobarbital, Phenytoin, Phosphorus, Prealbumin Procainamide, Progesterone, Prolactin, Quinidine, Rheumatoid Factor, SHBG, Sirollmus, STAT CK-MB, T4, Tacrolimus, Tacrolimus II, Testosterone, Tg, Theophylline, Theophylline II, TIBC, TIMP-1, Tobramycin, Total Billirubin, Total Estriol, Total Protein, Total PSA, Total T3, Total T4, Transferrin, Triglycerides, Troponin-1, Troponin-I ADV, TSH, T-Uptake, UIBC, Ultra HDL, Urea Nitrogen, Uric Acid, Urine/CSF Protein, Valproic Acid, Vancomycin, Vancomycin II, Vitamin D, antibodies against any of the foregoing, or any combination thereof.

### Receiving Member

According to an embodiment or the invention, the apparatus includes a receiving member, having an opening to receive a fluid sample, For example, the receiving member may be dimensioned to receive a fluid collector. In an embodiment of the invention, the receiving member may be in fluid communication with other components of the apparatus, for example at least one membrane test strip, sample retention member, and/or an Immunoassay-based fingerprint acquisition pad, through channels, for example tubes, piping, channels molded or carved into the apparatus, or any other suitable structure, made of any suitable material, for example plastic, ceramic, metal, glass, wood, rubber, polymer, fiber-reinforced polymer, or any combination thereof.

According to an embodiment of the invention, the channel or channels providing fluid communication between the components may have differing flow resistance, for example having channels, channel segments, or openings, that are narrower, wider, longer, or shorter than others, and/or having fluid paths with varying amounts of vertical rise or drop, such that the fluid channels within the device have varying degrees of flow resistance. For example, the channel that provides the fluid communication of the sample receiving member with the at least one membrane test strip may have greater flow resistance than the at least one channel that provides the fluid communication of the sample receiving member with the sample retention member, to ensure that a portion of the fluid sample is collected in the sample retention member.

In an embodiment of the invention, a single channel having multiple openings may connect the receiving member to each of the components of the apparatus with which it is in fluid communication, for example the at least one membrane test strip, sample retention member, and/or immunoassay-based fingerprint acquisition pad.

In an embodiment of the invention, the receiving member may include two or more chambers for receipt of a multi-pronged fluid collector, including but not limited to a dual-swab fluid collector. Components of the apparatus may be solely connected to one of the multiple chambers. For example, in a two chamber embodiment, one chamber may be solely connected to a sample retention member to ensure that a portion of the fluid sample is collected and stored without interaction of the other components of the apparatus.

An embodiment of the invention may accommodate fluids of varying viscosity, for example water, saliva, urine, blood, and liquids associated with genomics and proteomics. Generally, this is accomplished by varying the diameter of the channel or channels that provide the fluid communication of the sample receiving member with the other components of the apparatus, for example providing a wider channel diameter to accommodate a more viscous fluid.

In an embodiment of the invention, a channel dimensioned to be compatible with a fluid having the viscosity of water provides the fluid communication of the sample receiving member with the at least one membrane test strip and at least one channel dimensioned to be compatible with a fluid having the viscosity of water provides the fluid communication of the sample receiving member with the sample retention member.

In an embodiment of the invention, a channel dimensioned to be compatible with a fluid having the viscosity of urine provides the fluid communication of the sample receiving member with the at least one membrane test strip and at least one channel dimensioned to be compatible with a fluid having the viscosity of urine provides the fluid communication of the sample receiving member with the sample retention member.

In an embodiment of the invention, a channel dimensioned to be compatible with a fluid having the viscosity of saliva provides the fluid communication of the sample receiving member with the at least one membrane test strip and at least one channel dimensioned to be compatible with a fluid having the viscosity of saliva provides the fluid communication of the sample receiving member with the sample retention member.

In an embodiment of the invention, a channel dimensioned to be compatible with a fluid having the viscosity of blood provides the fluid communication of the sample receiving member with the at least one membrane test strip and at least one channel dimensioned to be compatible with a fluid having the viscosity of blood provides the fluid communication of the sample receiving member with the sample retention member. In an embodiment of the invention, a channel dimensioned to be compatible with a fluid having the viscosity of mucus provides the fluid communication of the sample receiving member with the at least one membrane test strip and at least one channel dimensioned to be compatible with a fluid having the viscosity of mucus provides the fluid communication of the sample receiving member with the sample retention member.

In an embodiment of the invention, a channel dimensioned to be compatible with a fluid having the viscosity of liquid associated with cell separation provides the fluid communication of the sample receiving member with the at least one membrane test strip and at least one channel dimensioned to be compatible with a fluid having the viscosity of liquid associated with cell separation provides the fluid communication of the sample receiving member with the sample retention member.

In an embodiment of the invention, a channel dimensioned to be compatible with a fluid having the viscosity of liquid biopsy, such as proteomics or genomics, provides the fluid communication of the sample receiving member with the at least one membrane test strip and at least one channel dimensioned to be compatible with a fluid having the viscosity of liquid biopsy provides the fluid communication of the sample receiving member with the sample retention member. Proteomics is the study of proteins. Genomics is a branch of molecular biology concerned with the structure, function, evolution, and mapping of genomes.

In an embodiment of the invention, the receiving member may have an inner surface, for example a lower surface, that an absorbent material, such as an absorbent material present in a fluid collector, may be compressed against, thereby expelling the fluid sample from the absorbent material. For example, the absorbent material may be compressed directly between a compression member present on the fluid collector and the lower surface of the receiving member or the receiving member may provide structural support to facilitate compression of the absorbent material between a compression member and the housing that at least partially surrounds the absorbent material.

### Sample Retention Member

According to an embodiment of the invention, the apparatus includes a sample retention member. The sample retention member may be used to securely contain a portion of the fluid sample, such as a split sample. The retained portion of the fluid sample may be used for further testing, for example for confirmation of a test result obtained using a membrane test strip, or to test for the presence or absence of other analytes in the fluid sample. The retained portion of the fluid sample may also be used for confirmation of the test subject's identity through analysis of a distinguishing feature thereof, including without limitation DNA, cells, proteomics, metals, and liquid biopsy.

According to one embodiment of the invention, the sample retention member includes an absorbent material, for example a pad or sponge, or made of woven or non-woven fibrous or fabric-like material, for example cellulose or a cellulose derivative, cotton, hydrophilic foam, wood pulp, polyvinyl alcohol fibers, or any combination thereof. The sample retention member may include an absorbent material that is part of the sample collection apparatus. The absorbent material may be surrounded by a barrier, such as a liquid-impermeable material, including without limitation plastic, ceramic, metal, glass, wood, rubber, polymer, fiber-reinforced polymer, or any combination thereof, to prevent the retained sample from leaking or evaporating. In an embodiment of the invention, the absorbent material may be removably attached to the apparatus to facilitate retrieval of the retained fluid sample. In an embodiment of the invention, the absorbent material may be accessed using a needle, for example by piercing a barrier surrounding the absorbent material. The retained sample may then be removed, for example, into a syringe attached to a needle, by means of withdrawal of the syringe to create suction.

According to an embodiment of the invention, the sample retention member includes a storage container defining a volume for storage of the fluid sample. In one embodiment of the invention, the sample retention member may be a vial made from a breakable or nearly unbreakable material, including without limitation glass, plastic, ceramic, metal, metal foil, wood, rubber, polymer, fiber-reinforced polymer, or any combination thereof. In an embodiment of the invention, the storage container may be accessed using a needle to pierce the wall of the storage container. For example, the storage container may include a pierceable member, such as a region of decreased wall thickness, and/or made of a soft, pierceable, or breakable material, including without limitation plastic, ceramic, metal, glass, metal foil, wood, rubber, polymer, fiber-reinforced polymer, or any combination thereof, that may be pierced. The retained sample may then be removed, for example, into a syringe attached to a needle, by means of withdrawal of the syringe to create suction. In an embodiment of the invention, the storage container may be removably attached to the apparatus, including without limitation, through a line of weakness that may allow the storage container to be broken free form the apparatus, through a threaded connection mechanism between the sample retention member and the fluid sample testing device, or through a twisting lock connection mechanism between the sample retention member and the fluid sample testing device.

According to an embodiment of the invention, the removable sample retention member may be linked to or coded consistently with the fluid sample testing device, including but not limited to, identical or related identification or serial numbers on both the sample retention member and the fluid sample testing device, identical or related bar code information on both the sample retention member and the fluid sample testing device, and the inclusion of radio frequency identification devices (RFID) on the sample retention member or the sample retention member and the fluid sample testing device. RFID incorporates the use of electromagnetic or electrostatic coupling in the radio frequency (RF) portion of the electromagnetic spectrum uniquely identify an object; such unique identification information may be information specific to the sample provider or information unique to the fluid sample testing device.

According to an embodiment of the invention, the sample retention member contains substances that facilitate a further use of the sample, including without limitation preservatives of stabilizers able to preserve sample integrity, for example substances able to inhibit microbial growth, kill microbes, prevent sample leakage, prevent sample evaporation, inhibit chemical or enzymatic degradation of substances in the sample, support survival of cells or other microbes in the sample, or any combination thereof.

According to an embodiment of the invention, the sample retention member may be bonded to a fingerprint acquisition pad. For example, such a bond may provide a safeguard against dissociation of the retained sample from the fingerprint.

According to an embodiment of the invention, the sample retention member may be in fluid contact solely with the sample receiving member and may not have any fluid contact with any other component of the apparatus.

The retained fluid sample may be used for further confirmation testing, including without limitation gas chromatography, liquid chromatography, mass spectrometry, liquid or gas chromatography with tandem mass spectrometry, polymerase chain reaction, DNA sequencing, Enzyme-Linked ImmunoSorbent Assay, Western Blotting, culturing for growth, or any combination thereof, using the retained fluid sample.

### Fluid Collector

In embodiment of the apparatus comprises a fluid collector for collecting a fluid sample. The present invention contemplates collecting a sample from a specific subject, such as a human subject, or testing environmental samples, such as testing air, water, soil, or some other substance, or a food or beverage, or a liquid extract of any of the foregoing for example, without limitation. The fluid collector is operative associated with the apparatus. The fluid collector may be removably associated with the apparatus/affixed to the apparatus, or comprise multiple units of which one or more is affixed or removably associated with the apparatus.

In an embodiment of the invention, the fluid collector includes an absorbent material or swab capable of absorbing a desired quantity of a fluid sample. The absorbent material may be made of any suitable material known to a person in the art, for example, without limitation, a pad or sponge, or woven or non-woven fibrous or fabric-like material, including without limitation cellulose or a cellulose derivative, cotton, hydrophilic foam, wood pulp, polyvinyl alcohol fibers, or any combination thereof. In an embodiment of the invention, the fluid collector includes a compression member, able to compress the absorbent material, that may be used to expel air from the absorbent material prior to collection of the fluid sample and/or encourage the fluid sample to flow into the absorbent material by creating suction as the compressed absorbent material returns to the uncompressed state. A compression member may also be used, for example, to compress the absorbent material and expel a fluid sample contained therein.

In one embodiment of the invention, the fluid collector includes multiple collection swabs. For example, a two-prong fluid collector with dual swabs may be implemented to collect the sample. In one embodiment, each swab of a multi-swab fluid collector may be selected based upon the specific swab collection characteristics. For example, in a dual-swab fluid collector, each swab may contain a material to assist in the collection of different samples such as the collection of different cell material.

A sufficiency indicator on the collector is contemplated. For example without limitation, a color indicator may either appear or disappear when a sufficient sample has been collected, for example when a sufficient volume has been absorbed to reach the location in the absorbent material where the sufficiency indicator is disposed. According to an embodiment of the invention, the sufficiency indicator may be operatively associated with the absorbent material and may protected from direct contact with the source of the fluid sample by a barrier, such as a transparent barrier, for example plastic or glass, such that the fluid sample will only reach the sufficiency indicator by passing into the absorbent material.

The sufficiency indicator color may be in the shape of a word or symbol that appears or disappears when a sufficient sample has been collected. For example, the sufficiency indicator may a diffusible dye, wherein dilution of the dye by the fluid sample causes a color to disappear, indicating that a sample of sufficient volume has been collected. In an embodiment of the invention, a combination of a non-diffusible and diffusible dye may be used together, such that the non-diffusible dye remains and provides an informative message when the diffusible dye disappears, for example the diffusible dye may form the letters “in” in the word “insufficient” such that the non-diffusible dye remains and forms the word “sufficient” when a sufficient sample has been collected.

The sufficiency indicator may be a pH-sensitive substance that changes color when the sample is encountered. For example, multiple pH sensitive indicators responding to different pH values may be preset, such that a color change is observed whether the sample is acidic, basic, or neutral. According to an embodiment of the invention, a pH-changing substance, such as an acid or base, may be disposed within the absorbent material, such that the sample will be of the correct pH to elicit the desired color change in the sufficiency indicator.

A closure member may be used. The closure member is capable of sealing the open end of a sample receiving member when the fluid collector is inserted into the open end of a sample receiving member. For example, the closure member may be dimensioned to fit closely in the opening in the open end of the receiving member, and the closure member or the open end of the receiving member may include a compressible material, including without limitation natural rubber such as vulcanized rubber, synthetic rubber such as neoprene or nitrite rubber, plastic, ceramic, or any combination thereof, disposed at the interface between the closure member and the opening in the open end of the sample receiving member, capable of creating a seal, such as an airtight or a watertight seal, when the sample receiving member receives the fluid collector.

After the fluid collector has been inserted into the sample receiving member, a device for securing the fluid collector within the sample receiving member is contemplated. The means for securing may prevent removal of the fluid collector from the sample receiving member after it has been inserted therein. The means for securing the fluid collector within the sample receiving member may include at least one projection extending from the fluid collector that cooperates with the at least one projection located on the inner surface of the sample receiving member, where such projections may include for example at least one locking tab and/or at least one annular ring. According to an embodiment of the invention, a closure member on the fluid collector may form a sufficiently secure closure as to constitute means for securing the fluid collector within the sample receiving member.

The sample receiving member may also include a tamper-evident seal, such that attempting to tamper with the contents of the apparatus will result in a visual indicator, for example by tears or breakage visible in an imprinted seal, for example tape or adhesive-backed foil having characters, symbols or a signature on a surface. Such a tamper-evident seal may be placed on the apparatus before its use, to create a visual confirmation that the intents of the apparatus have not been altered via the open end of the receiving member prior to testing, or after its use, to create a visual confirmation that the contents of the apparatus have not been altered via the open end of the receiving member subsequent to testing. According to an embodiment of the invention, the means for securing the fluid collector within the sample receiving member may constitute a tamper evident seal, in that attempted removal of the fluid collector from the sample receiving member after it has been inserted therein may result in visible damage to the apparatus.

According to an embodiment of the invention, the fluid collector includes a handle, for example made of wood, plastic, ceramic, or metal, and disposed, for example, at the end distal to the absorbent material. The handle may be removably attached, for example through an interference fit, adhesive, glue, or epoxy, that breaks or separates when the handle is twisted and/or pulled, or by a structure that allows the handle to be broken away, for example, a line of weakness.

The fluid collector may include a housing that at least partially surrounds the absorbent material. The housing may have multiple openings to allow the fluid sample to be absorbed by and expressed from the absorbent material. The openings in the housing may contain filtration members able to strain particulates from the fluid sample, resulting in reduction of the number of particulates that enter the absorbent material. The fluid collector may include a compression member able to compress the absorbent material against the housing. For example, the housing may be slideably coupled to a compression member with the absorbent material disposed between the compression member and an inner surface of the housing, such that the absorbent material may be compressed by movement of the compression member towards an inner surface of the housing. An embodiment of the invention includes means for securing the absorbent material in the compressed state, including without limitation cooperating threads, projections, and/or grooves operatively associated with the compression member and the housing. The absorbent material may be released from the compressed state before, concurrently with, or after encounter with the fluid sample, facilitating entry of the fluid sample into the absorbent material as the absorbent material returns to the relaxed state, creating suction. For example, the absorbent material may be operatively associated with a spring, such that compression of the absorbent material results in compression of the spring, and when compression is released the spring assists return of the absorbent material to the uncompressed state.

In an embodiment of the invention, the fluid collector is operatively associated with the lid of a fluid container including without limitation a urine cup. For example, the absorbent material may be disposed on the inner side of the lid, such that attachment of the lid to the fluid container results in contact between the absorbent material and a fluid sample. In certain embodiments of the invention, a portion of the fluid collector including the lid may be removably associated with a portion of the fluid collector including the absorbent material, allowing the absorbent material to be separated from the lid. The operative association of the fluid collector with the lid may include means for arresting the rotation of part of the fluid collector relative to the lid, including without limitation cooperating projections present on one member and grooves or slots present on the other member, for example to facilitate release of means by which the absorbent material is fixed in the compressed state.

### Saliva Producing Substances

Use of a saliva producing substance is contemplated by the present invention. Saliva producing substances elicit or increase saliva production in the test subject. For example, without limitation, the saliva producing substance may sugars, salts, acids, or any combination thereof. In an embodiment of the invention, the saliva producing substance may be associated with a fluid collector, for example located on or in the absorbent material or the housing. In an embodiment of the invention, the saliva producing substance may be separated from the fluid collector, for example in the form of a gum, candy, or powder, for administration to the test subject before, during or after the fluid collector is inserted into the test subject's mouth.

For example, without limitation, the sugar may be a monosaccharide, a disaccharide, a trisaccharide, an oligosaccharide, a polysaccharide, acarbose, allose, altrose, amylose, arabinose, calibiose, cyclodextrin, alph-cyclodextrin, beta-cyclodextrin, gamma-cyclodextrin, deoxyglucose, dextrin, dihydroxyacetone, erythrose, erythrulose, ficoli, fructo-oligosaccharides, fructose, galacto-oligosaccharides, galactose, gentiobiose, glucoasmine, glucose, glyceraldehyde, glycogen, gulosse, idose, inositol, inulin, isomaltose, lactose, lyxose, maltose, maltosvl-cvclodextrin, malt-trifose, mannan-oligosaccharides, mannoheptulose, marinose, melexltose, monnitol, psiccae, raffinose, ribitol, ribose, ribulose, sedoheptulose, sorbitol, sorbose, sucrose, tagatose, talose, threose, trehalose, xylose, xylulose, or any combination thereof.

For example, without limitation, the salt may an inorganic salt, organic salt, acid salt, alkali salt, neutral salt, or amino acid salt, or any combination thereof. The salt may include a cation and an anion, for example without limitation thereto, the cation may be aluminum, ammonium barium, beryilium, calcium, cesiu, chromium(II), chromium(III), chromium(IV), cobalt(II), cobalt(III), copper(I), copper(II), copper(III), gallium, helium, hydrogen, hydronium, iron(II), iron(III), lead(II), lead(IV), lithium, magnesium, manganese(II), manganese(III), manganese(IV), manganese(VII), nickel(I), nickel(III), nitronium, potassium, pyridinium, silver, sodium, strontium, tin(II), tin(IV), zinc, or any combination thereof, and an anion may be acetate, amide, tartrate, borate, bromate, bomide, carbonate, chlorate, chloride, chlorile, chromate, citrate, cyanate, dichormate, dihydrogen phosphate, fluide, formate, glutamate, hydride, hydrogen carbonate, hydrogen oxalate, hydrogen phosphate, hydrogen sulfate, hydrogen sulfite, hydroxide, hypobromite, hypochlorite, Iodate, iodide, nitrate, nitride, nitrite, oxalate, oxide, perchlorate, permanganate, peroxide, phosphate, phosphide, phosphite, pyrophosphate, sulfate, sulfide, sulfite, telluride, thiocyanate, thiosulfate, or any combination thereof. For example, according to an embodiment of the invention, the salt may be sodium chloride or potassium chloride.

The acid may be any suitable acid known to a person skilled in the art, for example acetic acid, acrylic acid, adipic acid, alginic acid, alkanesulfonic acid, amino acid, asorbic acid, benzoic acid, boric acid, butyric acid, carbonic acid, carboxylic acid, citrio acid, fattys acid, folic acid, formic acid, fumaric acid, gluconic acid, hdyriodic acid, hydrobromic acid, hydrochloric acid, hydroquinosulfonic acid, isoascorbic acid, lactic acid, maleic acid, malio acid, malonic acid, methanesulfonic acid, nitric acid, oxalic acid, p-toluenesulfonic acid, para-bromophenylsulfonic acid, phosphoric acid, propionic acid, salicyclic acid, stearic acid, succinic acid, sulfuric acid, tannic acid, tartaric acid, thioglycolic acid, toluenesulfonic acid, uric acid, or any combination thereof.

### Fingerprint Identification

An embodiment of the present invention includes a fingerprint pad to provide identification of an individual associated with the test, such as the test subject, test administrator, and/or one or more witnesses. The fingerprint pad may employ any suitable fingerprinting methodology, for example, without limitation, ink-based, immunoassay-based, electronic, semi-inkless, or inkless. In an embodiment of the invention, the fingerprint pad may be able to collect multiple fingerprints, for example having multiple fingerprint pads, having one fingerprint pad of sufficient size to accommodate multiple fingerprints, or having an electronic fingerprint pad.

The fingerprint pad may be an ink-based fingerprint pad. An embodiment of the invention includes a dispenser able to dispense an ink that can elicit a signal in the ink-based fingerprint pad. The fingerprint pad may also be inkless or semi-inkless, for example requiring no ink or compatible with an activator that appears transparent on the subject's skin, is readily cleaned off the subject's skin, or readily disappears, for example, when the subject's hands are rubbed together. According to an embodiment of the invention, the inkless fingerprint pad may be immunoassay-based, for example as described within U.S. Pat. No. 6,352,663 to Raouf A. Guirguis, issued Mar. 5, 2002 (the “863 patent”), and U.S. Pat. No. 5,244,815 to Raouf A. Guirguis, issued Sep. 14, 1993 (the “'815 patent”), which are incorporated herein by reference in their entirety. The immunoassay-based fingerprint pad may or may not be in fluid communication with a sample receiving member. Other embodiments of the invention may incorporate various features of the embodiments disclosed within the '863 and '815 patents. In embodiment of the invention having an inkless or semi-inkless fingerprint pad that requires an activator to elicit a signal, the apparatus may also include a dispenser to dispense the activator. According to an embodiment of the invention, the fingerprint pad may have a surface, such as an absorbent or adhesive surface, able to gather sweat, oils, and/or skin cells when a finger is pressed against it, that may require further processing to permit clear visualization of the fingerprint.

According to an embodiment of the invention, an inkless fingerprint pad may be an electronic fingerprint pad, including without limitation an optical scan fingerprint reader or a solid-state fingerprint reader. An embodiment of the invention includes a memory element, including without limitation volatile or non-volatile memory, for example a hard disk, floppy disk, magnetic tape, optical disk, flash memory, holographic memory, EEPROM, RAM, DRAM, SDRAM, or SRAM coupled to the fingerprint pad for storage of one or more fingerprints. According to an embodiment of the invention, the electronic fingerprint pads may have electrically charged surface elements, wherein portions of the surface are electrically discharged upon contact with the finger surface, such as the ridges of the finger surface, such that the fingerprint is recorded in the pattern of discharged elements, whereby the fingerprint pattern may be stably stored within the surface for a time after it is created until it is read, for example through connection of the apparatus with an external device, including without limitation a base station. An embodiment of the invention include means of transmission of the captured fingerprint, for example to an external device or network, including without limitation through a hard-wired connection, for example employing wires, cables, or a docking station or docking connector, for employing a connection including without limitation USB, IEEE 1394, serial, parallel, or SCSI, or a wireless connection, for example employing infrared, RF. IEEE 802.11, Bluetooth, IEEE 802.15, or Wi-Fi.

In an embodiment of the invention, a cover encloses the fingerprint acquisition pad. The cover may be secured using various mechanisms, for example, without limitation, a tab-and-slot connector, latch, spring latch, adhesive tape, or security tape. The cover may be secured prior to fingerprint acquisition and/or after fingerprint acquisition.

The foregoing description provided background, features, implements and processes usable in combination with the specific improvements and refinements that will be further described in greater detail hereinafter.

Previously described embodiments (e.g., FIG. 21A to FIG. 24C) include lateral flow or other types of analyte test strips in a cassette or in a stem of a swab (in various forms). Such strips include sandwich and competitive binding strips, reaction pad strips such for alcohol (the reaction pad turning colors depending on the concentration of alcohol present, such as those employing a solid-phase chemistry using a preferably highly specific enzyme reaction. All such strips are within the scope of the present invention (strips). See previous discussion regarding such strips, which is applicable here (e.g., FIGS. 21A to 24).

Applicant has determined, however, that improved results can be obtained from such test strips inserted into a swab stem via a swab stem strip holder, which has a tip (preferably rigid or at least semi-rigid) that can engage with sponge material on the tip of the swab, and improve fluid migration from the sponge into the one or more test strips held in place by such swab stem strip holder. Such a swab stem strip holder also helps protect the strips during assembly and also improves the assembly process.

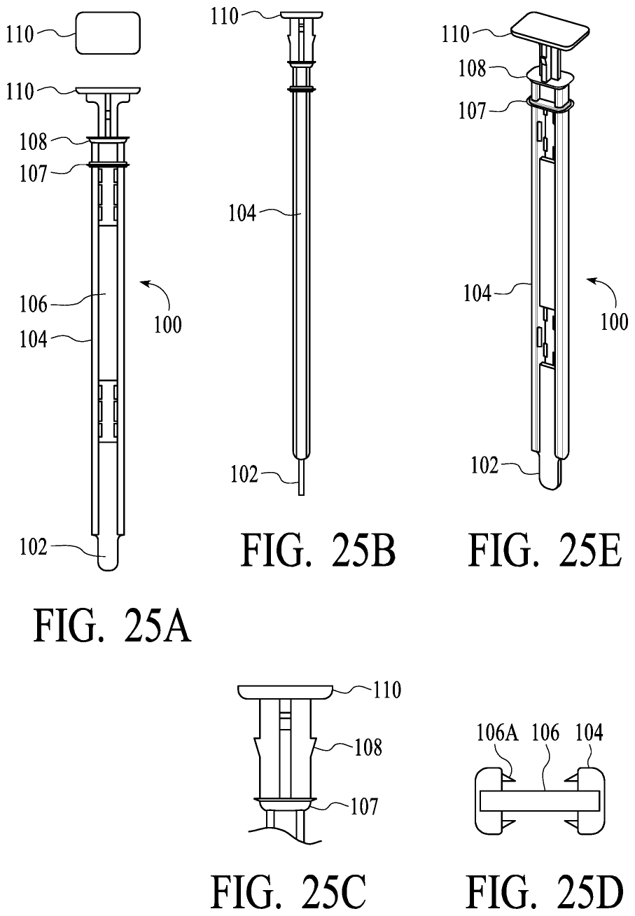

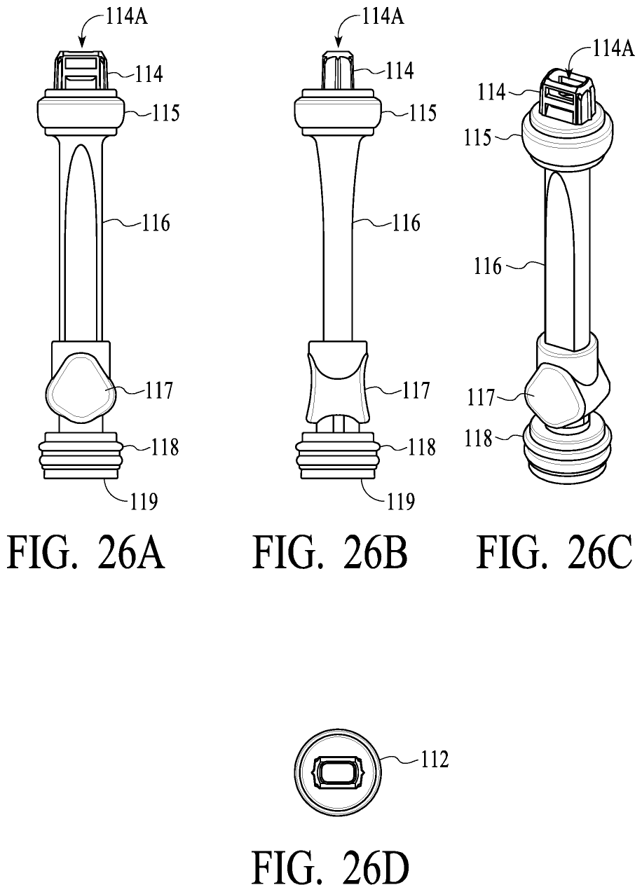

Referring now to FIGS. 25A to 25 E, details of an exemplary swab stem strip holder will now be described. Strip holder 100 preferably consists of a plastic material such as poly carbonate PC or general purpose polystyrene GPPS or similar materials, and is rigid or semi-rigid. Walls 104 and floor 106 define a channel for holding the test strip, which may be in a back-to-back configuration as illustrated (there being two channels, one on each of opposing sides of strip holder 100, such that strips may be held in a back-to-back configuration. Strip holder 100 also preferably includes one or a plurality of blade or wiper type seals 107, 108 that may slidably engage surfaces of the swab (see swab stem 116 of FIGS. 26A to 26C) to form a seal, which preferably is air and water tight (or substantially air and water tight). A top structure 110 of strip holder 100 rest on/in a correspondingly shaped top of the opening of swab stem 116 into which strip holder 100 is inserted. The channel of strip holder 100 preferably includes a plurality of protrusions 106A that engage with strips inserted into strip holder 100 and reduce the tendency of such strips to slide downward in the channel (such strips preferably being cut to extend from the top of the channel into to lower extremity of strip holder 100). Strip holder 100 also preferably has extending member 102 that extends (downward in FIG. 25A for example) beyond the channel defined by walls 014 and floor 106 and is dimensioned to engage the sponge of the swab (see, e.g. swab/sponge material as described elsewhere herein, such as 12A. 12, etc.). Extending member 102 preferably extends farther in the downward direction that the test strips to engage the swab/sponge material and compress it inward such as the test strips lower ends are not bent substantially by the swab/sponge material. Improved engagement by extending member 102 help fluid flow (e.g., saliva) into the sample pad of the test strip (see, e.g., FIGS. 21A-C and FIG. 2 for more exemplary details of such test strips).

FIGS. 26A-26D illustrate an exemplary preferred embodiment of swab stem 116 into which strip holder 100 is inserted. At the top of swab stem 116 is swab upper portion 114 having opening 114A at the uppermost portion thereof, which is an entry into an interior volume of swab stem 116 into which strip holder 100 is positioned. Upper seal 115, rest implement 117 and wiper-type seals 118 preferably are provided as illustrated, and preferably are an elastomeric-type overmold, such as previously described herein. Bottom portion 119 provides an open space into which the swab/sponge material is inserted and attached preferably by an adhesive. It should be noted here that extending member 102 is dimensioned in length so that it protrudes from the bottom of the interior volume of swab stem into the open space of bottom portion 119 such as it engages the swab/sponge material such as previously described. Swab stem top 112 is provided to preferably snap onto the top of swab stem 116 to provide a more expansive surface area for a user to push the swab stem down such as in a compression tube as described herein.

FIG. 31 illustrates and exploded view of such an exemplary preferred embodiment utilizing strip holder 100. Swab step top 112 engages with swab stem 116, preferably after strip holder 100 holding one or more test strips (see FIGS. 21A-21C and FIG. 22 and other discussion herein for exemplary details of such test strips) in inserted. Top structure 110 of strip holder 100 preferably engages the uppermost portion of swab stem 116 at opening 114A, and blade or wiper type seals 107, 108 slidably engage interior surfaces of swab stem 116 so that air and fluid do not pass from the interior volume of swab stem 116 through the opening at 114A. Swab/sponge 12A is illustrated adhered to the bottom portion of swab stem 116, such as described herein. Extending member 102 engages swab/sponge 12A, which test strips in strip holder are in fluid communication with fluid absorbed into swab/sponge 12A.

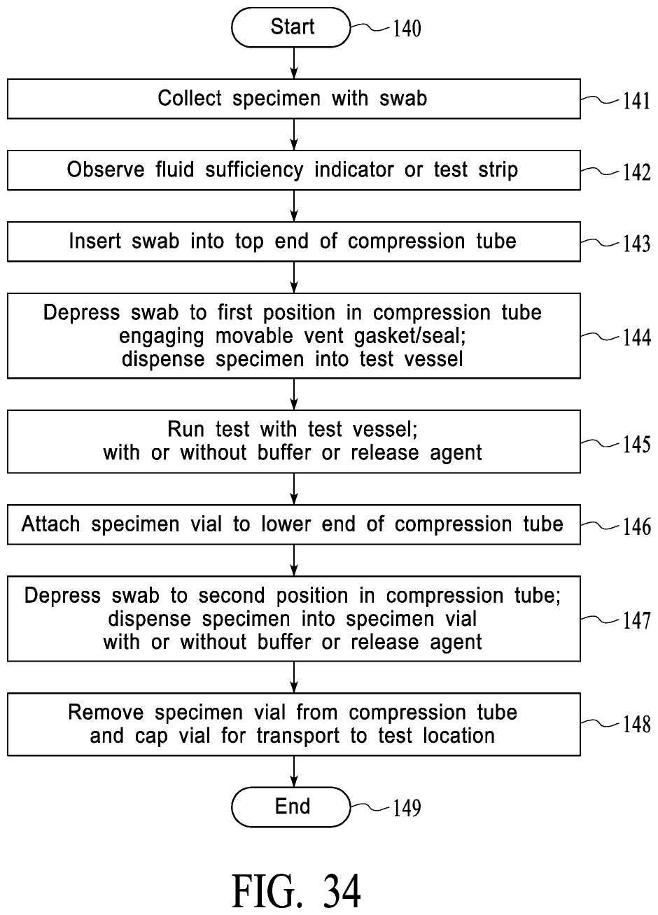

While in such embodiments one or more rapid test results may be viewed via one or more test strips and one or more windows in swab stem 116 as previously described, in some instances it is desired that remaining specimen be captured in a specimen vial/container/vessel, such as vial 32′ as illustrated and also as previously described. Preferably, swab/sponge 12A is compressed by insertion of the swab into compression tube 54, where it engages preferably movable venting gasket/seal 121, 122, 123 (which may also be gasket 30 such as described in connection with FIGS. 16B and 16C). This may be processes such as those described in connection with FIG. 34, or it may be direct compression to release fluid into specimen container 32′ (which may be a vial or other container that connects to the end of compression tube 54 such as by way of threads 54A (see FIG. 30) and threads 54A′. Optionally, specimen container 32′ after receipt of the fluid specimen may be remove (e.g., unscrewed) from compression tube 54 and be sealed by cap 124, which preferably is a water/air tight lid such as a threaded cap as illustrated.

As will be appreciated based on the description herein, in such embodiments, strip holder 100 holds one or more test strips, each of which may test for one or more analytes or substances, and it may be inserted into a test swab and secured with one or more seals, and be in fluid communication with swab/sponge material receiving the fluid specimen, and optionally provide a specimen via compression with a movable venting gasket/seal in a specimen container such as for lab analysis. Such a strip holder/swab assembly may be used in various of the embodiment describe herein, including Single Swab Devices and Dual Swab Devices, and combinations of such embodiments will be apparent to those of skill in the art based on the description herein.

It should also be understood that embodiments of the present invention are not limited to the particular exemplary structure identified herein. As illustrative additional examples, instead of the flat strip holder having two channels, it should be readily understood that the swab stem may be enlarged to accommodate a triangle tube structure (triangle in cross section) have three strip holding channels, or a square tube structure (square in cross section) having four strip holding channels, or a pentagon tube structure (five sided in cross section) having five strip holding channels, etc. Strip dimensions may be modified along with swab channel dimensions to accommodate various strip holding structures having n (n being an integer from 1 to 10). What is important is that the strip holder be insertable into the swab stem and engage the absorbent sponge-like material, and enable fluid communication between the sponge-like material and the sample pad of the test strips.

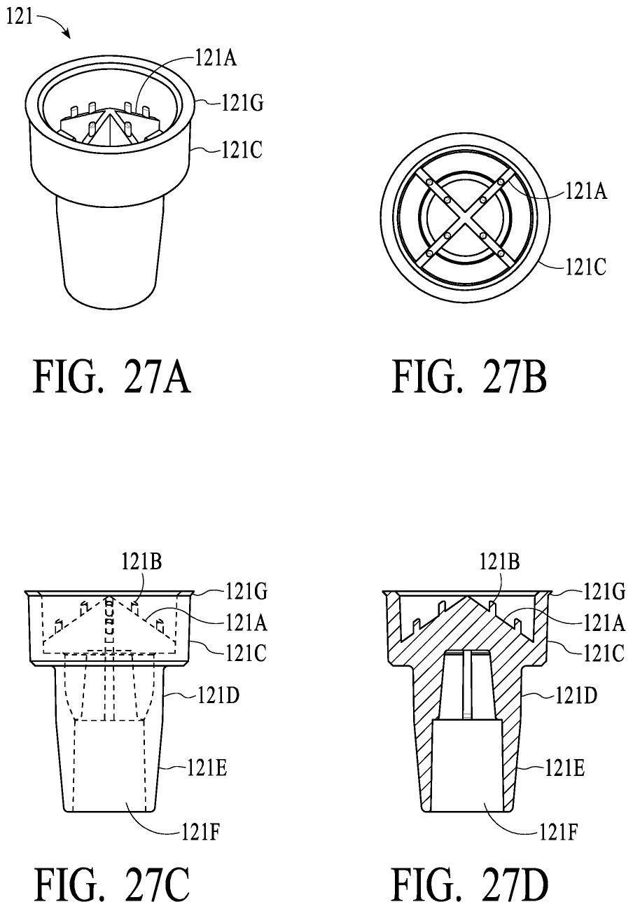

Referring now to FIGS. 27A to 29D, improved movable venting gasket/seals 121, 122 and 123 will now be described. As described herein, improvements in compressing fluid specimen swabs are highly desired in the industry. Efficiency of fluid collection and extraction, and improvements in extracting materials from the fluid, are important in producing testing products and services with improved accuracy, efficiency, sensitivity, etc.

Applicant has discovered improved ways to vent extract fluid specimens from a swab/sponge using a compression tube, wherein a movable venting gasket/seal a through hold or cavity into which the swab/sponge is inserted to create compression, and the venting movable venting seal/gasket has a one and preferably a plurality of cross members traverse to the direction of swab/sponge insertion. Preferably such cross members have one and preferably a plurality of projections that engage with, and penetrate and distort fibers or other material of the swab/sponge, thereby facilitating fluid release from the swab/sponge, and improved fluid and target analyte substance recovery from the fluid in the swab/sponge.

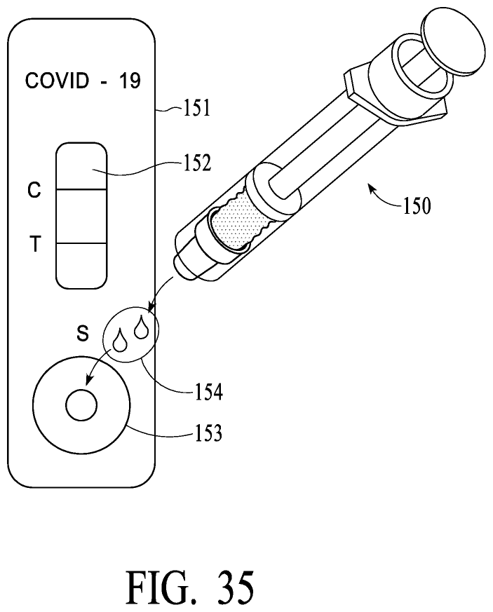

FIGS. 27A-27D illustrate a first one of such embodiments. As illustrated movable venting gasket/seal 121 is preferably a round initially cylindrical (having body 121C and upper sealing lip 121G, serving to engage with and provide a sliding seal with a compression tube), with a middle section 121D, and a lower section 121E. As described elsewhere herein, such a movable venting gasket/seal engages with a compression tube and provides compression of an inserted swab, while air may be vented vial a vent path created between the compression tube and a specimen container/vial connected to the compression tube (see, e.g., FIGS. 9, 10 14, 15, 16A and 16B and related and other/similar description). In one illustrated embodiment, cross members 121A connect at a center point that is higher than the portion where cross members 121A connect with body 121C. As illustrated, cross members 121A have an upward slope toward the cylindrical center of body 1210 such that the center portion of the cross members will engage a downwardly inserted swab/sponge initially before the swab/sponge engages the lower point of the cross members (e.g., where cross members connect to body 121C. One or plurality of projections 121B extend upwardly to also engage the swab/sponge. Middle section 121D is dimensioned in diameter such as to be inserted into the specimen container or vial, and create a horizontal ledge as illustrated (and as described elsewhere herein) to create a seal with the specimen container or vial. Importantly with such improved embodiments, lower section 121E of movable venting gasket/seal is tapered in overall circumference but also has an open interior space 121F (cross members 121A do not extend downward into interior space 121F). Preferably, lower section 121E is dimensioned so that it extends from the bottom of the compression tube when in its initial position when first engaging the swab/sponge. Also preferably, lower section 121E is tapered and creates a dropper-type opening so that drops of fluid may be dispensed from open volume 121F prior to a specimen container or vial being connected to the compression tube. This allows the swab/compression tube/movable venting gasket/seal to extract and dispense fluid from the swab/sponge much like a syringe or dropper, such as into a test vessel or cassette (see, e.g., FIGS. 34 and 35 and related description).

What is important is, movable venting gasket/seal engages the swab/sponge in a first position that enables air to vent, with the movable venting gasket/seal being dimensioned to extend from the compression tube to provide droplets of fluid such as into a test vessel or cassette, while having cross members and preferably upwardly extending projections, protrusions or spikes to engage with, compress, distort and other manipulate the swab/sponge so as to more efficiently release fluid and target analytes or other materials in the fluid from the swab/sponge; at a second lower sealing position, the venting mechanism is closed, and the fluid is sealed in the specimen container or vial (such as described elsewhere herein).

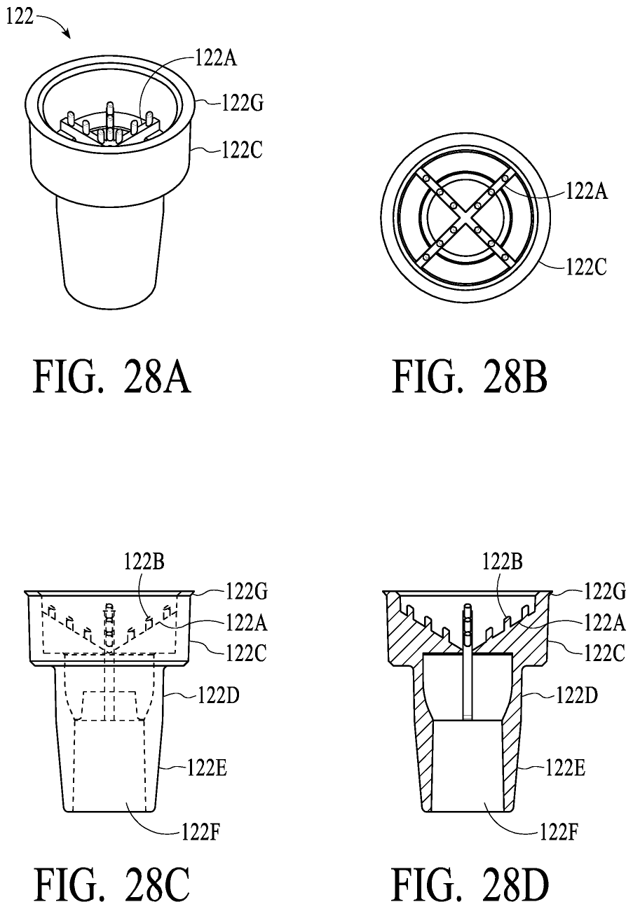

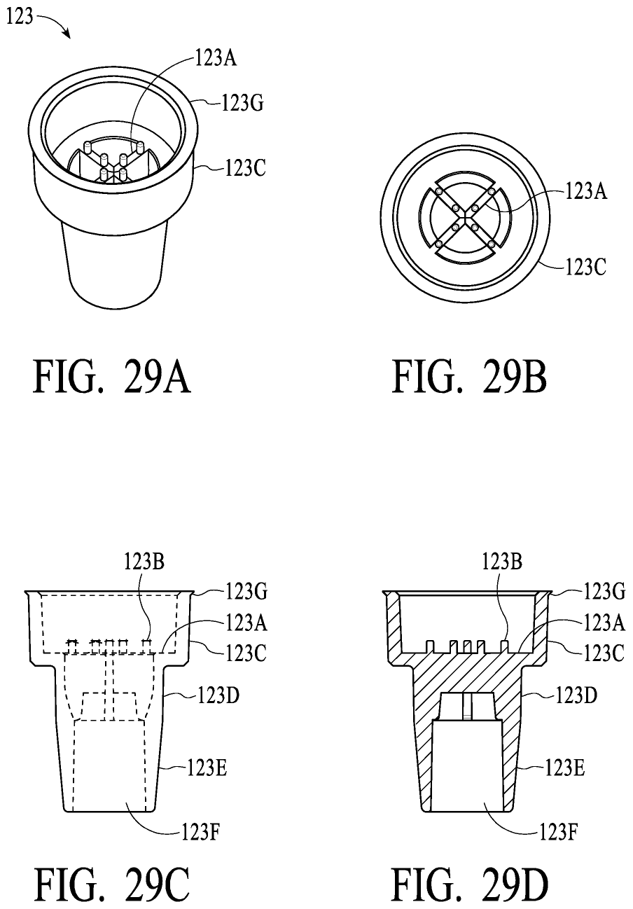

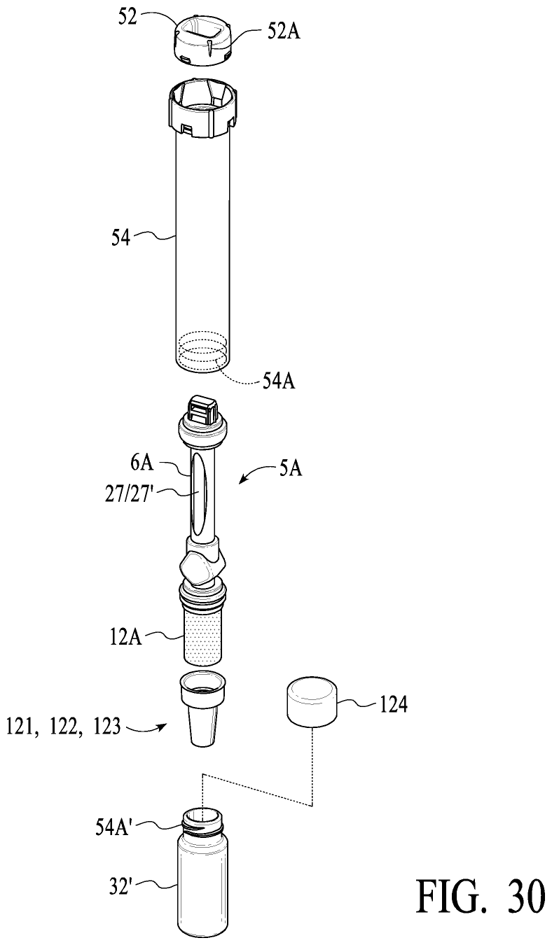

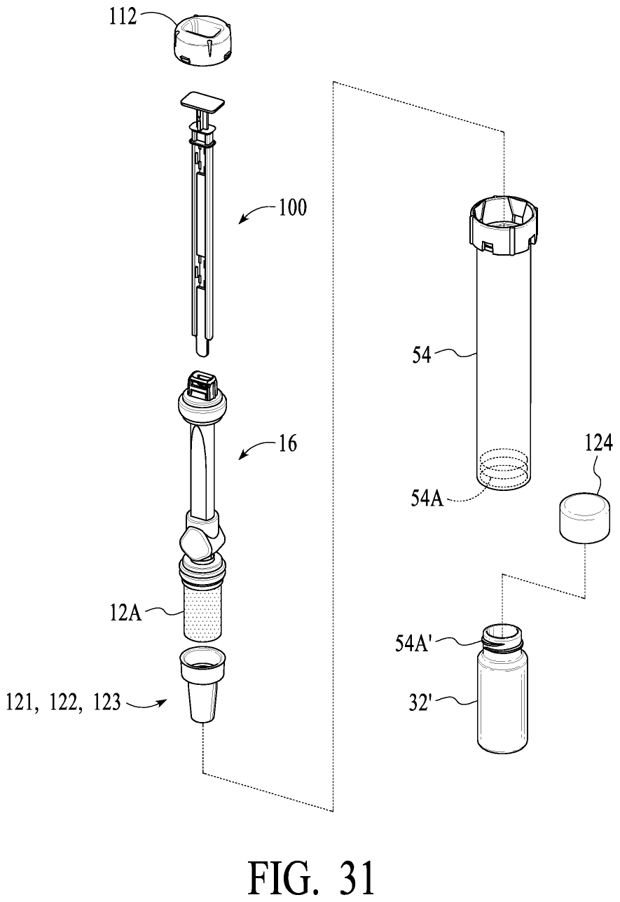

Alternative preferred embodiments are illustrated in FIGS. 28A-28D and FIGS. 29A-29D (for ease of discussion, corresponding numerals and letters are used with respect to the corresponding elements of the embodiments in FIGS. 27A-27D, 28A-28D and 29A-29D.

In the embodiment of FIGS. 28A-28D, cross members 122A of movable venting gasket/seal 122 descend downwardly such that they connect in a center portion that is lower than where cross members 122A connect with body 122C. Thus, an inserted swab/sponge will first engage cross member 122A at an outer circumferential portion of the swab/sponge as compared to an interior portion of the swab/sponge. Also as illustrated, projections 122B preferably are provided to further engage with, compress, distort and manipulate the portion of the swab/sponge engaging with these portions of cross members 122A. For ease of discussion, the function of movable venting gasket/seal 122 is similar to that of movable venting gasket/seal 121, and such description is incorporated herein by reference and not repeated.

In the embodiment of FIGS. 29A-29D, cross members 123A of movable venting gasket/seal 123 extend such that they connect in a center portion that is substantially the same height where cross members 123A connect with body 123C. Thus, an inserted swab/sponge will first engage cross member 123A just at the cross member surfaces while other portions of the swab/sponge are not engaging the cross member surfaces. Also as illustrated, projections 123B preferably are provided to further engage with, compress, distort and manipulate the portion of the swab/sponge engaging with these portions of cross members 123A. For ease of discussion, the function of movable venting gasket/seal 123 is similar to that of movable venting gasket/seal 121, and such description is incorporated herein by reference and not repeated.

As will be appreciated those of skill in the art based on the description herein, but as further reflected in FIGS. 30, 31 (previously described), 32A, 32B, 33A and 33B, such embodiments of the improved movable venting gasket/seal are desirably utilized in both Single Swab Devices and Dual Swab Devices. For example, a Single Swab Device is further illustrated in FIG. 30. The discussion of FIG. 20 is incorporated herein by reference as is the discussion above with respect to FIGS. 27A-29D. Such embodiments advantageously utilize one of movable venting gaskets/seals 121, 122, or 123, and also may provide a workflow as described in connection with FIGS. 34 and 35, with cap 124 provided for specimen container/vial 32′.

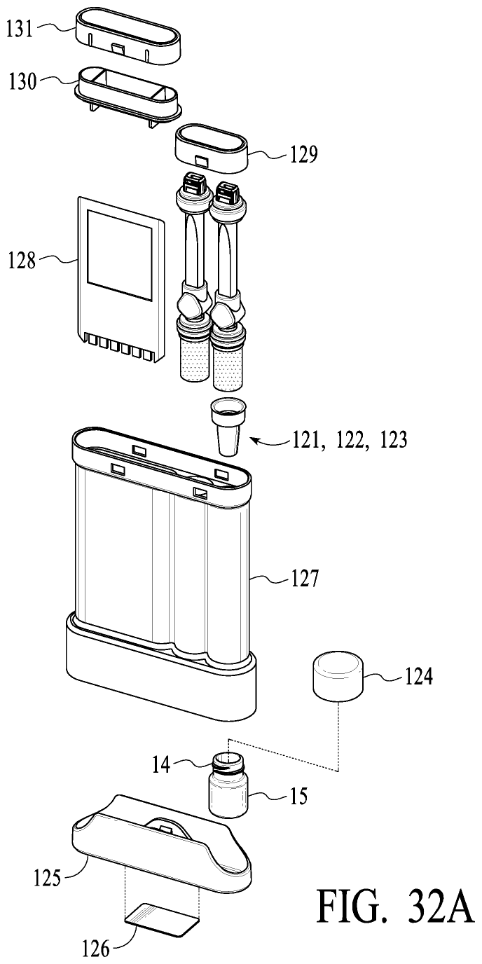

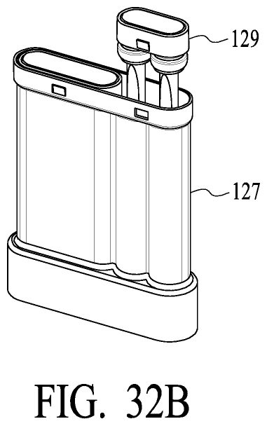

A Dual Swab Device 127 is illustrated in FIG. 32A, having dual compression tubes as described elsewhere herein, cassette 128 having one or a plurality of analyte test strips, sealing gasket 130 and top cap 131, and a dual swab assembly generally denoted here as 129. Also generally denoted here are specimen container/vial 15 with threads 14, bottom cap 125, forensic seal 126 which must be punctured or removed to remove bottom cap 125 (making the device tamper-evident as forensic seal 126 must be punctured or removed to remove bottom cap 125 and get access to specimen container/vial 15. Cap 124 is provided for workflows in which the specimen container/vial is removed and desired to be re-sealed (such as previously described). FIG. 32B illustrates swab assembly 129 deeply but not fully depressed into the body of Dual Swab Device 127. Such embodiments advantageously utilize one of movable venting gaskets/seals 121, 122, or 123, and also may provide a workflow as described in connection with FIGS. 34 and 35, with cap 124 provided for specimen container/vial 32′.

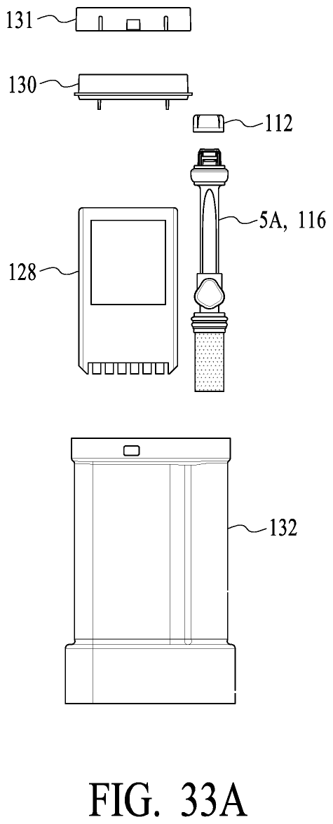

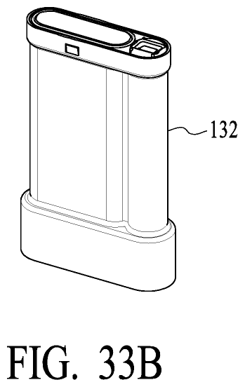

A Single Side Device 132 having a cassette-side only (no vial side) is illustrated in FIGS. 33A and 33B. Device 132 has a single compression tube as described elsewhere herein, cassette 128 having one or a plurality of analyte test strips, sealing gasket 130 and top cap 131, and a single swab assembly generally denoted here as 5A, 116 (with swab top cap illustrated as 112. It is noted here that swab 116 desirably utilizes strip holder 110 as described herein with reference to FIGS. 25A-26C, and such description of such embodiments is incorporated herein, but not repeated for ease of discussion. FIG. 33B illustrates the swab assembly fully depressed into the body of Single Swab Device 132.

As will appreciated by those of skill in the art based on the description herein, advantageous workflows are enabled by the embodiments described herein. Several exemplary workflows are described in connection with FIGS. 34 and 35.

FIG. 34 is a flow chart describes various methods in accordance with such embodiments. The flow starts at step 140. Various preparation steps may occur, such as registering a patient/donor with a paper requisition or a portal or app/smartphone registration process. Typically patient/donor information is captured for identification of the specimen to be collected as part of the workflow. At step 141, a specimen is collected with a swab, which may include as a preferable example a swab to collect oral fluid such as oral swabs described herein, included swabs with one or a plurality of test strips in the stem of the swab such as swabs with integral test strips as described in connection with FIGS. 25A-26C. At step 142, the patient/donor or an observer may monitor the specimen collector by way of the test strips in swab stem, or alternatively a saliva/fluid sufficiency indicator strip such as described herein. What is important is that the swab become sufficiently saturated with fluid for the desired test, and the test strip or sufficiency indicator strip may be used to make such assessment. At step 143, with the swab sufficiently saturated for the desired test(s) to be performed, the swab may be inserted into a compression tube. Swab insertion into a compression tube has been described in connection with various embodiments herein, and is particularly applicable to embodiment described in connection with FIGS. 27A-29D and FIGS. 30-31. At step 144, the swab is inserted to a first position in the compression tube where it engages the movable vent gasket/seal, and based on such insertion compression of the swab/sponge material occurs and fluid (e.g., oral fluid) is dispensed from the open end of the lower tip of the movable vent gasket/seal (see, e.g., description of embodiments described in connection with FIGS. 27A-29D). With the lower tip of the movable vent gasket/seal extending from the bottom of the compression tube, one or more drops of fluid may be desirably dispensed like a dropper or syringe into a test vessel (this may be a vessel that actually runs a test or a vessel from which specimen is taken to run a test). What is important is that the swab may be inserted to a desired point to dispense fluid from the end of the movable vent gasket/seal. At step 145, a test may optionally be run with the test vessel, which may be a direct run with a test strip or other test mechanism, which may include a buffer or release agent. For example, the test vessel may contain a lateral flow test strip as described, or an antigen (Ag) test strip such as for detected a virus such as COVID. The test vessel may have an Ag release agent such as described herein. Various such test vessels that may conduct a test using a fluid specimen dispensed in this manner are used in embodiments of the present invention. If no further testing is desired, the flow may end at this point. Also, an initial test may already have been run with test strips in the swab stem, so testing via the test vessel may be a second level of testing with this specimen.

At step 146, a specimen container or vial may be connected to the end of the compression tube, such as by threads or other means such as described herein. With the specimen container or vial connected to the compression tube, at step 147 the swab may be further depressed deeper into the compression tube, thereby dispensing additional specimen into the specimen container or vial, which may be empty (neat specimen collection) or may contain a liquid or sold buffer or release agent. In one particular embodiment, a surfactant material containing a detergent (such as described herein) is dried on the inside surface of the specimen container or vial, which is washed or released from the inside surface of the specimen container or vial so that it may act on the specimen (such breaking down cellular walls, releasing antigens, etc.). What is important is that the specimen container or vial contains a buffer/release agent, or none at all, based on the tests to be conducted on the collected specimen. It is further noted that, at step 147, the movable vent gasket/seal preferably is at its lowest position and effectively seals the specimen in the specimen container or vial and impedes specimen from escaping out of the compression tube (e.g., out of the vent path) and impedes specimen from moving back up the compression tube. At step 148, the specimen container or vial may be removed (e.g., unscrewed) from the compression tube and capped such for transport to a laboratory or other test facility. Also, the specimen container or vial may used for additional testing, such as placing a test strip into the vial, dispensing specimen from the specimen container or vial into another test vessel, etc. At step 149, this workflow ends.

As an additional example of how such embodiments may be utilized is described in connection with FIG. 35. As illustrated, a swab assembly after a patient's/donor's specimen has been collected has been inserted into a compression tube, generally illustrated as 150. This generally correlates to the first position of step 144 of FIG. 34. As illustrated, drops of specimen 154 may be dispense from the movable vent gasket/seal extending from the compression tube, as described elsewhere herein and as generally illustrated by 150. Such drops 154 may be dispensed into an exemplary test vessel 151, which may be test for a disease like COVID or for one or more drugs or other analytes. In the illustrated exemplary embodiment, a lateral flow test strip 152 is illustrated, which may of a type of strip as described herein. Typically, drops 154 are dispensed into a well 153 of test vessel 151 (which may be a cassette type structure holding one or more test strips). Well 153 may be single part well or a dual part well into which a buffer or release agent may be dispensed in a first part and the drops may be dispensed into a second part, such as the two parts may be mixed before the mixed specimen/release agent are exposed to the test strip. What is important is that drops may be dispensed from the movable vent gasket/seal into a test vessel, such as described in the exemplary process flow of FIG. 34.

Although the invention is described herein with reference to specific embodiments, various modifications and changes can be made without departing from the scope of the invention as set forth in the claims below. The combination of embodiments is expressly anticipated, unless the embodiments and specifically mutually exclusive. A claimed invention may include multiple embodiments as disclosed herein. Accordingly, the specification and figures are to be regarded in an illustrative rather than a restrictive sense, and all such modifications are intended to be included within the scope of the invention. Any benefits, advantages, or solutions to problems that are described herein with regard to specific embodiments are not intended to be construed as a critical, required, or essential feature or element of any or all the claims.

From time-to-time, the invention is described herein in terms of these example embodiments. Description in terms of these embodiments is provided to allow the various features and embodiments of the invention to be portrayed in the context of an exemplary application. After reading this description, it will become apparent to one of ordinary skill in the art how the invention can be implemented in different and alternative environments. Unless defined otherwise, all technical and scientific terms used herein have the same meaning as is commonly understood by one of ordinary skill in the art to which this invention belongs.

The preceding discussion is presented to enable a person skilled in the art to make and use the invention. The general principles described herein may be applied to embodiments and applications other than those detailed below without departing from the spirit and scope of the invention as defined by the appended claims. The invention is not intended to be limited to the embodiments shown, but is to be accorded the widest scope consistent with the principles and features disclosed herein.

In addition, while a particular feature of the invention may have been disclosed with respect to only one of several embodiments, such feature may be combined with one or more other features of the other embodiments as may be desired. It is therefore, contemplated that the claims will cover any such modifications or embodiments that fall within the true scope of the invention.

The various diagrams may depict an example architectural or other configuration for the invention, which is done to aid in understanding the features and functionality that can be included in the invention. The invention is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the invention. In addition, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one”, “one or more” or the like; and adjectives such as “conventional”. “traditional”, “normal”, “standard”, “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

A group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the invention may be described or claimed in the singular, the plural is contemplated to be within the scope thereof unless limitation to the singular is explicitly stated.

The presence of broadening words and phrases such as “one or more”, “at least”. “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed across multiple locations.

Unless stated otherwise, terms such as “first” and “second” are used to arbitrarily distinguish between the elements such terms describe. Thus, these terms are not necessarily intended to indicate temporal or other prioritization of such elements.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the field or any related fields are intended to be within the scope of the following claims.

One skilled in the art will recognize that different embodiments may be formed in a similar manner having different characteristics depending upon need, performance, or some other criteria. It will thus be appreciated by those skilled in the art that changes could be made to the embodiments described above without departing from the broad inventive concept thereof. It is understood, therefore, that the invention disclosed herein is not limited to the particular embodiments disclosed, but it is intended to cover modifications within the spirit and scope of the present invention as defined by the appended claims.

Although this invention has been described in conjunction with specific embodiments thereof, many alternatives, modifications and variations will be apparent to those skilled in the art. Accordingly, the embodiments of the invention as set forth herein are intended to be illustrative, not limiting. Various changes may be made without departing from the true spirit and full scope of the invention as set forth herein.

Although the invention has been described in conjunction with specific preferred and other embodiments, it is evident that many substitutions, alternatives and variations will be apparent to those skilled in the art in light of the foregoing description. Accordingly, the invention is intended to embrace all of the alternatives and variations that fall within the spirit and scope of the appended claims. For example, it should be understood that, in accordance with the various alternative embodiments described herein, various systems, and uses and methods based on such systems, may be obtained. The various refinements and alternative and additional features also described may be combined to provide additional advantageous combinations and the like in accordance with the present invention. Also as will be understood by those skilled in the art based on the foregoing description, various aspects of the preferred embodiments may be used in various subcombinations to achieve at least certain of the benefits and attributes described herein, and such subcombinations also are within the scope of the present invention. All such refinements, enhancements and further uses of the present invention are within the scope of the present invention.

What is claimed is:

1. A method for testing a fluid, comprising the steps of:
collecting a fluid specimen via a swab having a compressible absorbent tip;
inserting the swab in a compression tube to a first position, wherein compression of the absorbent tip occurs and a first portion of the collected specimen dispensed into an external vessel via a movable vent gasket/seal, wherein an end portion of the movable vent gasket/seal extends beyond an end of the compression tube;
appending a specimen container onto the end of the compression tube, where the end portion of the movable vent gasket/seal extends into the specimen container;
moving the swab to a second position deeper in the compression tube, wherein a second portion of the collected specimen dispenses into a specimen container.

2. A method for testing a fluid, comprising the steps of:
collecting a fluid specimen via a swab having a compressible absorbent tip;
inserting the swab in a compression tube to a first position, wherein compression of the absorbent tip occurs as the swab contacts a movable vent gasket/seal, wherein the movable vent gasket/seal comprises a cylindrical upper portion having one or more cross members impeding movement of the absorbent tip but allowing passage of fluid specimen into a vessel connected to an end portion of the compression tube; and
depressing the swab further into the compression tube to a second position, wherein during movement from the first position to the second position, air vents from the compression tube and vessel, wherein at the second position the movable vent gasket/seal seals fluid in the vessel.

3. A test swab, comprising:
a swab stem having an absorbent tip on a bottom end of the swab stem, the swab stem having an interior cavity open from a top end of the swab stem to the bottom end of the swab stem;
a test strip holder adapted to hold one or more analyte test strips, each such test strip testing or one or more analytes, wherein the test strip holder is adapted to slide into the top end of the swab stem and upon full insertion into the swab stem seals the interior cavity of the swab stem, wherein the test strip holder has extending member that upon full insertion into the swab stem engages the adsorbent tip on the bottom end of the swab stem;
one or more analyte test strips positioned in one or more channels of the test strip holder, wherein upon full insertion of the test strip holder into the swab stem the test strips are in fluid communication with the absorbent tip;
wherein the one or more analyte test strips contact fluid absorbed by the absorbent tip and provide one or more test results via a transparent portion of the swab stem.
